(19) United States
(12) Patent Application Publication
(10) Pub. No.: US 2021/0188883 A1
(43) Pub. Date: Jun. 24, 2021

(54) HYDROPHILIC SILANES

(71) Applicants: Dow Silicones Corporation, Midland, MI (US); Dow Corning Toray Co., Ltd., Tokyo (JP)

(72) Inventors: Michael Salvatore Ferritto, Midland, MI (US); Lenin James Petroff, Bay City, MI (US); Tatsuo Souda, Chiba (JP)

(21) Appl. No.: 16/080,461

(22) PCT Filed: Feb. 21, 2017
(86) PCT No.: PCT/US2017/018613

§ 371 Date: Aug. 28, 2018

(51) Int. Cl. C07F 7/18; C08K 9/06; C08K 5/54; C08K 3/22; A61K 8/58; A61Q 17/04; C09C 1/04; C09D 183/06

(52) U.S. Cl. CPC C07F 7/1804; C08K 9/06; C08K 5/5403; C08K 3/22; C08K 2003/2296; A61Q 17/04; C09C 1/043; C09D 183/06; A61K 8/585

(57) ABSTRACT

A composition comprising: an organosilane having formula (I) (I) X-A-Z, wherein X is —SiR⁴ₙR²₍₃₋ₙ₎ wherein each R⁴ is independently OR¹ or halogen, wherein each R¹ in independently hydrogen or C₁₋₄ hydrocarbyl and each R² is independently C₁₋₄ hydrocarbyl, and n is from 1 to 3, A is C₁₋₁₀ hydrocarbylene, wherein the backbone of the hydrocarbylene is substituted with one or more oxygen atoms, one or more nitrogen atoms, or carbonyl, Z is a sugar group, a monoglycerol group, a diglycerol group, a polyglycerol group, or a xylitol group, and wherein the composition is a personal care composition, surface treating composition, an antifog composition, a coating composition, a surface treated powder, a paint composition, or an ink composition.

### CROSS REFERENCE TO RELATED APPLICATIONS

This application claims the benefit of U.S. Provisional Patent Application No. 62/328,129 filed 27 Apr. 2016 under 35 U.S.C. § 119 (e). U.S. Provisional Patent Application No. 62/328,129 is hereby incorporated by reference.

### TECHNICAL FIELD

The present invention generally relates to a composition comprising organosilanes, wherein the composition is a personal care composition, surface treating composition, an antifog composition, a coating composition, a surface treating composition, a paint composition, or an ink composition. The present invention further relates to methods of treating surfaces with the surface treating compositions and to the treated surfaces.

### BACKGROUND

Silanes have been made by various methods including the direct process, hydrosilylation, and Grignard reactions. Silanes have a variety of known uses. For example, they can be used as monomers in making elastomers, polymers and resins, as coupling agents, additives for various compositions such as detergents, household and personal care formulations, and as surface treating agents for rendering surfaces hydrophilic. Some silanes have multiple uses in a variety of applications.

Silanes used for the treatment of surfaces to render the surfaces hydrophilic have known hydrophilic groups bound to the silicon atom of the silane. Examples of these hydrophilic groups are polyethylene oxide and polypropylene oxide. However, polyethylene oxide and polypropylene oxide have some unwanted properties. Similarly, compositions comprising silanes having polyethylene oxide and polypropylene oxide also have unwanted properties or do not provide desired performance properties.

We see a long-felt need in the industries for compositions comprising organosilanes that do not comprise either polyethylene oxide or polypropylene oxide. We think organosilanes not comprising either polyethylene oxide or polypropylene oxide but having hydrophilic groups may enable greater formulation latitude in providing better compatibility, and may have improved performance in cosmetic, paint, ink, surface treating, skincare, sun care, hair care, antifog, and coating compositions. Further, we see a need for silanes to render surfaces hydrophilic and/or improve dispersibility of surface treated powders in compositions such as aqueous compositions.

### SUMMARY OF THE INVENTION

The present invention is directed to a composition comprising an organosilane having formula (I) X-A-Z, wherein X is —SiR⁴ₙR²₍₃₋ₙ₎, wherein each R⁴ is independently OR¹ or halogen, wherein each R¹ in independently hydrogen or C₁₋₄ hydrocarbyl and each R² is independently C₁₋₄ hydrocarbyl, and n is from 1 to 3, A is C₁₋₁₀ hydrocarbylene, wherein the backbone of the hydrocarbylene is substituted with one or more oxygen atoms, one or more nitrogen atoms, or carbonyl, Z is a sugar group, a monoglycerol group, a diglycerol group, a polyglycerol group, or a xylitol group, and wherein the composition is a personal care composition, surface treating composition, an antifog composition, a coating composition, a surface treated powder, a paint composition, or an ink composition.

A method of treating a surface with the treating composition.

The compositions of the invention render surfaces hydrophilic, provide improved, dispersibility of powders, transparency, UV protection, contact angle, among other properties. The method of treating a surface renders the surface hydrophilic.

### DETAILED DESCRIPTION OF THE INVENTION

The Brief Summary and Abstract are incorporated here by reference. The invention embodiments, uses and advantages summarized above are further described below.

Aspects of the invention are described herein using various common conventions. For example, all states of matter are determined at 25° C. and 101.3 kPa unless indicated otherwise. All % are by weight unless otherwise noted or indicated. All % values are, unless otherwise noted, based on total amount of all ingredients used to synthesize or make the composition, which adds up to 100%. Any Markush group comprising a genus and subgenus therein includes the subgenus in the genus, e.g., in “R is hydrocarbyl or alkenyl,” R may be alkenyl, alternatively R may be hydrocarbyl, which includes, among other subgenuses, alkenyl. For U.S. practice, all U.S. patent application publications and patents referenced herein, or a portion thereof if only the portion is referenced, are hereby incorporated herein by reference to the extent that incorporated subject matter does not conflict with the present description, which would control in any such conflict.

Aspects of the invention are described herein using various patent terms. For example, “alternatively” indicates a different and distinct embodiment. “Comparative example” means a non-invention experiment. “Comprises” and its variants (comprising, comprised of) are open ended. “Consists of” and its variants (consisting of) is closed ended. “Contacting” means bringing into physical contact. “May” confers a choice, not an imperative. “Optionally” means is absent, alternatively is present.

Aspects of the invention are described herein using various chemical terms. The meanings of said terms correspond to their definitions promulgated by IUPAC unless otherwise defined herein. For convenience, certain chemical terms are defined.

The term “halogen” means fluorine, chlorine, bromine or iodine, unless otherwise defined.

The term “IUPAC” refers to the International Union of Pure and Applied Chemistry.

“Periodic Table of the Elements” means the version published 2011 by IUPAC.

A composition, the composition comprising: an organosilane having formula (I) X-A-Z, wherein X is —SiR⁴ₙR²₍₃₋ₙ₎, wherein each R⁴ is independently OR¹ or halogen, wherein each R¹ in independently hydrogen or C₁₋₄ hydrocarbyl and each R² is independently C₁₋₄ hydrocarbyl, and n is from 1 to 3, A is C₁₋₁₀ hydrocarbylene, wherein the backbone of the hydrocarbylene is substituted with one or more oxygen atoms, one or more nitrogen atoms, or carbonyl, Z is a sugar group, a monoglycerol group, a diglycerol group, a polyglycerol group, or a xylitol group, and wherein the composition is a personal care composition, a paint composition, a surface treating composition, an antifog composition, a coating composition, a surface treated powder, or an ink composition.

The composition comprises an organosilane having formula (I) X-A-Z, wherein X is —SiR⁴ₙR²₍₃₋ₙ₎, wherein each R⁴ is independently OR¹ or halogen, wherein each R¹ in independently hydrogen or C₁₋₄ hydrocarbyl and each R² is independently C₁₋₄ hydrocarbyl, and n is from 1 to 3, A is C₁₋₁₀ hydrocarbylene, wherein the backbone of the hydrocarbylene is substituted with one or more oxygen atoms, one or more nitrogen atoms, or carbonyl, Z is a sugar group, a monoglycerol group, a diglycerol group, a polyglycerol group, or a xylitol group. The hydrocarbyl groups represented by R¹ and R² typically have from 1 to 10 carbon atoms, alternatively from 1 to 6 carbon atoms, alternatively 1 to 4 carbon atoms, alternatively 1 to 3 carbon atoms, alternatively 1 or 2 carbon atoms, alternatively 2 to 6 carbon atoms, alternatively 2 or three carbon atoms. Acyclic hydrocarbyl groups containing at least three carbon atoms can have a branched or unbranched structure. Examples of hydrocarbyl groups include, but are not limited to, alkyl, such as methyl, ethyl, propyl, 1-methylethyl, butyl, 1-methylpropyl, 2-methylpropyl, 1,1-dimethylethyl, pentyl, 1-methylbutyl, 1-ethylpropyl, 2-methylbutyl, 3-methylbutyl, 1,2-dimethylpropyl, 2,2-dimethylpropyl, hexyl, heptyl, octyl, nonyl, and decyl; cycloalkyl, such as cyclopentyl, cyclohexyl, and methylcyclohexyl; aryl, such as phenyl and napthyl; alkaryl, such as tolyl and xylyl; arakyl, such as benzyl and phenethyl; alkenyl, such as vinyl, allyl, and propenyl; aralkenyl, such as styryl and cinnamyl; and alkynyl, such as ethynyl and propynyl.

Hydrocarbylene groups represented by A typically have from 1 to 10 carbon atoms, alternatively from 2 to 10 carbon atoms, alternatively from 1 to 6 carbon atoms, alternatively from 2 to 6 carbon atoms, alternatively from 1 to 3 carbon atoms, alternatively 2 or 3 carbon atoms, alternatively from 3 to 10 carbon atoms, alternatively from 3 to 6 carbon atoms, alternatively 3 carbon atoms, alternatively 6 carbon atoms, alternatively 10 carbon atoms. The backbone of the hydrocarbylene is substituted and the substitution comprises one or more oxygen atoms, one or more nitrogen atoms, or carbonyl. The hydrocarbylene group represented by A may be further substituted in addition to the substitution of the backbone.

“Substituted,” in reference to the backbone of the hydrocarbylene, means that one of the carbons of the carbon backbone is replaced by one or more atoms other than carbon or one or two carbonyl groups, alternatively one or more of O, N, or carbonyl, alternatively O, N, carbonyl, —NC(O)N—, —NC(O)O—, or —C(O)O—, alternatively O, N, carbonyl, —NC(O)N—, —NC(O)O. The substitution may be within the carbon chain or at an end of the carbon chain. For example, a hydrocarbylene comprising 3 carbon atoms and substituted with oxygen includes, but is not limited to, the following structures: —CH₂CH₂CH₂O— and —CH₂OCH₂CH₂—, and a hydrocarbylene having one carbon atom and substituted with oxygen means —CH₂O—.

“Substituted,” other than in reference to the backbone of the hydrocarbylene, means that a hydrogen atom of a hydrocarbyl or hydrocarbylene group is substituted with a group or atom other than hydrogen or carbon, alternatively a hydroxyl, amine or oxygen, wherein the oxygen is part of a carbonyl group.

Acyclic hydrocarbylene groups containing at least three carbon atoms can have a branched or unbranched structure Examples of hydrocarbylene groups with the backbone of the hydrocarbylene substituted and represented by A include, but are not limited to, diyl groups formed by removing two hydrogen atoms from an alkane, such as methane (e.g., 1,1-methane-diyl), ethane, propane, 1-methylethane, butane, 1-methylpropane, 2-methylpropane, 1,1-dimethylethane, pentane, 1-methylbutane, 1-ethylpropane, 2-methylbutane, 3-methylbutane, 1,2-dimethylpropane, 2,2-dimethylpropane, hexane, heptane, octane, nonane, and decane; from cycloalkane, such as cyclopentane (e.g., 1,3-cyclopentane-diyl), cyclohexane, and methylcyclohexane; from arene, such as benzene and naphthalene; from alkarene, such as toluene and xylene; from alkene, such as ethane, propene, phenyl butane; from an aralkene, such as styrene, and 3-phenyl-2-propene; and from alkyne, such as ethyne and propyne, and wherein one or more, alternatively from 1 to 3, alternatively 1 or two, of the carbons of the hydrocarbylene backbone is substituted with O, N, carbonyl, —NC(O)N—, or —NC(O)O; and further including eugenol,

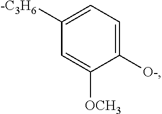

—(CH₂)ₐCH₂O—, —(CH₂)ₐCH₂OCH₂CH(OH)CH₂—, wherein a is from 0 to 6, alternatively from 1 to 3, alternatively 2.

The groups represented by Z include, but are not limited to, sugar group, a monoglycerol group, a diglycerol group, a polyglycerol group, or a xylitol group. The sugar groups may be one or more sugar groups, represented by the chemical formula C₆H₁₂O₆, linked together. Examples of sugar groups represented by Z include, but are not limited to, N-methyl glucosamine (e.g., (2R,3R,4R,5S)-6-(Methylamino)hexane-1,2,3,4,5-pentol) and glucose (e.g., D-glucose), where Z is bonded and/or linked through the nitrogen or an oxygen atom to A. In one embodiment the sugar group is N-methyl glucosamine, where the group is bonded and/or linked by the nitrogen atom to A. The monoglycerol, diglycerol and polyglycerol groups represented by Z comprise one (in the case of monoglycerol) two (in the case of diglycerol) or more glycerol units linked through an oxygen atom.

The monoglycerol, diglycerol or polyglycerol group is represented by Glyₐ, wherein Gly is R³CH₂CH(R³)CH₂R³, where each R³ independently represents hydroxyl, an oxygen atom linking to A, or an oxygen atom linking to another Gly unit, and a is an integer ≥1, alternatively an integer from 2 to 6, alternatively 2 or 3, alternatively 2, alternatively 3. In one embodiment, Glyₐ represents —OCH₂CH(OH)CH₂OH, —OCH₂CH(OH)CH₂OCH₂CH(OH)CH₂OH, —OCH(CH₂OCH₂CH(OH)CHOH)CH₂OCH₂CH(OH)CH₂OH, or —O(C3H6O2)_{b}H, wherein b is greater than or equal to 1, alternatively from 2 to 8, alternatively from 2 to 6, alternatively 2 or 3, alternatively 2 alternatively 3, alternatively Glyₐ represents —OCH₂CH(OH)CH₂OH, OCH₂CH(OH)CH₂OCH₂CH(OH)CH2OH, or —OCH(CH₂OCH₂CH(OH)CHOH)CH₂OCH₂CH(OH)CH₂OH.

The xylitol group is represented by Xyl, wherein Xyl is CH₂(R⁵)CH(R⁵)CH₂C(H)(R⁵)CH₂R⁵, where each R⁵ independently represents hydroxyl or an oxygen atom linking Xyl to A. An example of the xylitol group is —OCH₂CH(OH)CH₂CH(OH)CH₂(OH).

The group represented by X is —SiR⁴ₙR²₍₃₋ₙ₎, where each R⁴ is independently OR¹ or halogen, wherein each R¹ is independently hydrogen or C₁₋₁₀ hydrocarbyl and each R² is independently C₁₋₁₀ hydrocarbyl, and n is from 1 to 3, alternatively 2 or 3, alternatively 2, alternatively 3, alternatively 1. Acyclic hydrocarbyl groups containing at least three carbon atoms can have a branched or unbranched structure. Examples of hydrocarbyl groups represented by R¹ include, but are not limited to, alkyl, such as methyl, ethyl, propyl, 1-methylethyl, butyl, 1-methylpropyl, 2-methylpropyl, 1,1-dimethylethyl, pentyl, 1-methylbutyl, 1-ethylpropyl, 2-methylbutyl, 3-methylbutyl, 1,2-dimethylpropyl, 2,2-dimethylpropyl, hexyl, heptyl, octyl, nonyl, and decyl; cycloalkyl, such as cyclopentyl, cyclohexyl, and methylcyclohexyl; aryl, such as phenyl and napthyl; alkaryl, such as tolyl and xylyl; aralkyl, such as benzyl and phenethyl; alkenyl, such as vinyl, allyl, and propenyl; aralkenyl, such as styryl and cinnamyl; and alkynyl, such as ethynyl and propynyl. Examples of hydrocarbyl groups represented by R² are those described from R¹ above. In one embodiment R¹ is the same as R² which is methyl, alternatively ethyl.

Examples of —SiR⁴ₙR²₍₃₋ₙ₎ include, but are not limited to, trimethoxysilyl, triethoxysilyl, tripropoxysilyl, methyldimethoxysilyl, ethyldiethoxysilyl, ethyldimethoxysilyl, methyldiethoxysilyl, dim ethylmethoxysilyl, diethyldiethoxysilyl, diethylmethoxysilyl, dimethylethoxysilyl.

Examples of organosilane having formula (I), X-A-Z (I) include, but are not limited to, the following: 3-glycerol-propyltrimethoxysilane,

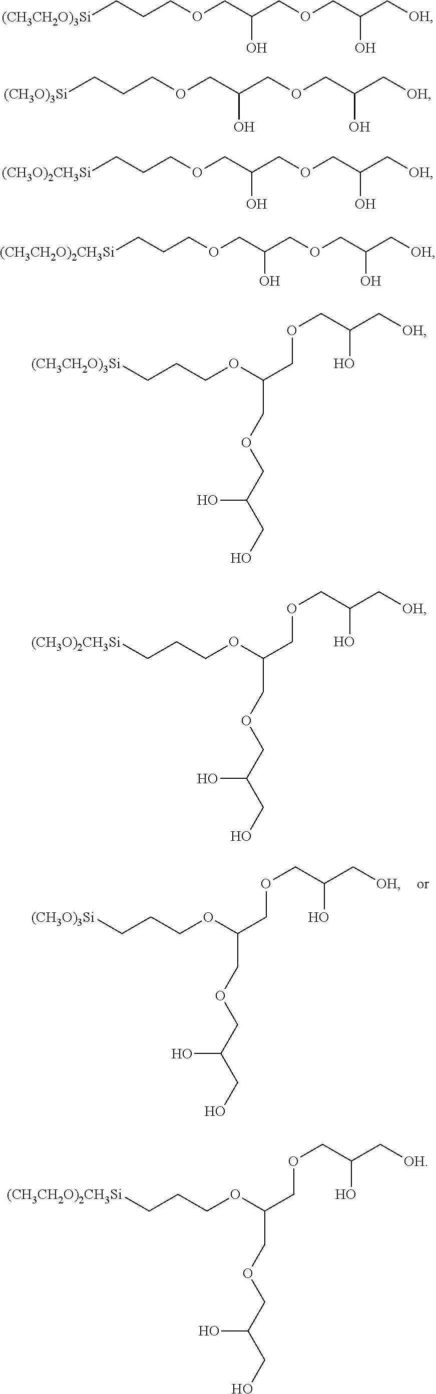

and those where A-Z is

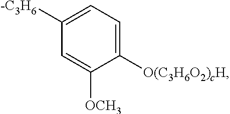

where c is ≥1, alternatively 1-5 alternatively 2-4, alternatively 2, alternatively 3, and X is trimethoxysilyl, triethoxysilyl, tripropoxysilyl, methyldimethoxysilyl, ethyldiethoxysilyl, ethyldimethoxysilyl, methyldiethoxysilyl, dimethylmethoxysilyl, diethyldiethoxysilyl, diethylmethoxysilyl, or dimethylethoxysilyl.

One embodiment of the invention is a composition comprising the organosilane described above. A “composition,” with respect to the organosilane is the organosilane itself and one additional material. Example of additional materials include solvents, surfactants, additives, acids, bases, oils, emollients, waxes, conditioners such as cationic, amphoteric, and betaine conditioning agents, opacifiers, suncreens, and metal oxides.

A method for preparing an organosilane, the method comprising reacting an organic compound Z¹-E¹, wherein Z is a sugar, a monoglycerol group, a diglycerol, a polyglycerol, or a xylitol group, E¹ is hydroxyl, amine or an organic group comprising a reactive functional group, where the reactive functional group comprises hydroxyl, amine, oxirane, or isocyanate, with an organic compound D¹-E², wherein D¹ is an organic group comprising an unsaturated hydrocarbyl group having 2 to 12 carbon atoms, and E² is a reactive functional group comprising hydroxyl, amine, oxirane, or isocyanate, at a temperature and pressure sufficient to cause Z¹-E¹ and D¹-E² to react, to form F¹, wherein F¹ is an intermediate, and reacting F¹ with an organosilane of formula Si(OR¹)ₙ(R²)₃₋ₙH, where R¹ is an alkyl group containing 1 to 4 carbon atoms and R² is an alkyl group containing 1 to 4 carbon atoms, n is from 1 to 3, and a hydrosilylation catalyst.

Z¹ represents a sugar, a monoglycerol group, a diglycerol group, a polyglycerol group, or a xylitol group, alternatively a diglycerol group, a polyglycerol group, or a xylitol, alternatively a diglycerol, a triglycerol group, or xylitol group. The sugar represented by Z¹ is as described above for the organosilane. In one embodiment, the sugar is glucose (D-glucose), fructose, or N-methyl glucamine, alternatively N-methylglucamine, alternatively D-glucose or N-methyl glucamine.

Monoglycerol, diglycerol and triglycerol groups represented by Z¹ are represented by the formula Glyₐ, wherein Gly is R³CH₂CH(R³)CH₂R³, where each R³ independently represents hydroxyl, an oxygen atom linking to E¹, or an oxygen atom linking to another Gly unit, and a is an integer ≥1, alternatively a is an integer ≥2, alternatively an integer from 2 to 6, alternatively 2 or 3, alternatively 2, alternatively 3. In one embodiment, Glyₐ represents —OCH₂CH(OH)CH₂OH, —OCH₂CH(OH)CH₂OCH₂CH(OH)CH₂OH, —OCH(CH₂OCH₂CH(OH)CHOH)CH₂OCH₂CH(OH)CH₂OH, or —O(C₃H₆O₂)_{b}H, where b is greater than 1, alternatively greater than 2, alternatively from 2 to 8, alternatively from 2 to 6, alternatively 2 or 3, alternatively 2 alternatively 3, alternatively Glyₐ represents —OCH₂CH(OH)CH₂OCH₂CH(OH)CH2OH, or —OCH(CH₂OCH₂CH(OH)CHOH)CH₂OCH₂CH(OH)CH₂OH.

The xylitol group represented by Z¹ has the formula CH₂(R⁵)CH(R⁵)CH₂C(H)(R⁵)CH₂R⁵, where each R⁵ independently represents hydroxyl or an oxygen atom linking to E¹. In one embodiment, the xylitol group is —OCH₂CH(OH)CH₂C(H)(OH)CH₂OH.

E¹ is hydroxyl, amine or an organic group comprising a reactive functional group, where the reactive functional group comprises hydroxyl, amine, oxirane, or isocyanate. The amine group represented by E¹ typically is a primary or secondary amine, alternatively a primary amine. The group bonded to the secondary amine is typically a hydrocarbyl group having from 1 to 10 carbon atoms, alternatively 1 to 6 carbon atoms, alternatively 1 carbon atom. Examples of hydrocarbyl groups of the secondary amine include, but are not limited to, alkyl, such as methyl, ethyl, propyl, 1-methylethyl, butyl, 1-methylpropyl, 2-methylpropyl, 1,1-dimethylethyl, pentyl, 1-methylbutyl, 1-ethylpropyl, 2-methylbutyl, 3-methylbutyl, 1,2-dimethylpropyl, 2,2-dimethylpropyl, hexyl, heptyl, octyl, nonyl, and decyl; cycloalkyl, such as cyclopentyl, cyclohexyl, and methylcyclohexyl; aryl, such as phenyl and napthyl; alkaryl, such as tolyl and xylyl; aralkyl, such as benzyl and phenethyl; alkenyl, such as vinyl, allyl, and propenyl; aralkenyl, such as styryl and cinnamyl; and alkynyl, such as ethynyl and propynyl.

The organic group comprising a reactive functional group represented by E¹ comprises hydroxyl, amine, oxirane, or isocyanate and typically comprises a hydrocarbyl group having from 1 to 10 carbon atoms, alternatively from 1 to 6 carbon atoms, alternatively 1 to 3 carbon atoms, wherein the hydrocarbyl group is substituted with the hydroxyl, amine, oxirane, or isocyanate. Examples of hydrocarbyl groups of the organic group comprising a reactive functional group include, but are not limited to, alkyl, such as methyl, ethyl, propyl, 1-methylethyl, butyl, 1-methylpropyl, 2-methylpropyl, 1,1-dimethylethyl, pentyl, 1-methylbutyl, 1-ethylpropyl, 2-methylbutyl, 3-methylbutyl, 1,2-dimethylpropyl, 2,2-dimethylpropyl, hexyl, heptyl, octyl, nonyl, and decyl; cycloalkyl, such as cyclopentyl, cyclohexyl, and methylcyclohexyl; aryl, such as phenyl and napthyl; alkaryl, such as tolyl and xylyl; aralkyl, such as benzyl and phenethyl; alkenyl, such as vinyl, allyl, and propenyl; aralkenyl, such as styryl and cinnamyl; and alkynyl, such as ethynyl and propynyl.

Examples of the organic group comprising a hydroxyl group represented by E¹ include, but are not limited to, hydroxyalkyl, such as hydroxymethyl, hydroxyethyl. hydroxypropyl, hydroxybutyl, hydroxypentyl, hydroxyhexyl, hydroxyl dodecyl.

The amine comprised by the organic group comprising a reactive functional group is as defined for E¹ above. The oxirane group of the organic group comprising a reactive functional group is a hydrocarbyl group having oxirane functionality. As used herein, “oxirane” means a compound in which an oxygen atom is directly attached to two adjacent carbon atoms of a carbon chain or ring system ((i.e, a three member cyclic ether), and is represented by the following structural formula —CH(O)CH₂. Example of the organic group comprising an oxirane functional group include, but are not limited to, alkenyl oxide, such as ethenyl oxide, propenyl oxide, 1-butenyl oxide, 1-pentenyl oxide, 1-hexenyl oxide, 1-septenyl oxide, 1-octenyl oxide; and cycloalkenyl oxide, such as cyclohexenyl oxide.

The isocyanate group has the structure —N═C═O, where the isocyanate may be bonded or linked through the nitrogen atom directly to Z¹ or part of a larger organic group comprising the isocyanate group. Examples of the organic group comprising isocyante include alkyl isocyanates, such as methyl, ethyl, propyl, 1-methylethyl, butyl, 1-methylpropyl, 2-methylpropyl, 1,1-dimethylethyl, pentyl, 1-methylbutyl, 1-ethylpropyl, 2-methylbutyl, 3-methylbutyl, 1,2-dimethylpropyl, 2,2-dimethylpropyl, hexyl, heptyl, octyl, nonyl, and decyl, wherein the alkyl group is substituted with an isocyanate group; cycloalkyl, such as cyclopentyl, cyclohexyl, and methylcyclohexyl, wherein the cycloalkyl group is substituted with an isocyanate group; aryl, such as phenyl and napthyl wherein the aryl group is substituted with an isocyanate group; alkaryl, such as tolyl and xylyl, wherein the alkaryl group is substituted with an isocyanate group; aralkyl, such as benzyl and phenethyl, wherein the aralkyl group is substituted with an isocyanate group; alkenyl, such as vinyl, allyl, and propenyl, wherein the alkenyl group is substituted with an isocyanate group; aralkenyl, such as styryl and cinnamyl, wherein the aralkenyl group is substituted with an isocyanate group; and alkynyl, such as ethynyl and propynyl, wherein the alkynyl group is substituted with an isocyanate group.

Examples of the organic compound Z¹E¹ include, but are not limited to, 2,3-epoxypropyldiglycerol, 2,3-epoxypropyltriglycerol, 2,3-epoxypropylpolyglycerol, N-2,3-epoxypropyl-N-methylglucamine, 3-aminopropyldiglycerol, 3-aminopropyltriglycerol, 3-aminopropylpolyglycerol, N-3-aminopropyl-N-methylglucamine, 3-isocyanatopropyldiglycerol, 3-isocyanatopropyltriglycerol, 3-isocyanatopropylpolyglycerol, N-3-icocyanatopropyl-N-methylglucamine, glycerol, diglycerol, triglycerol, polyglycerol, and N-methylglucamine. Compounds according to formula Z¹E¹ may be purchased commercially or synthesized from readily available starting materials using reactions known in the art. For example, methods of synthesizing these materials can be found in Japanese Patent documents JP2001-261672 A1 and JP2004-277548 A1, both of which are hereby incorporated by reference for their teaching related to synthesizing compounds according to the formula Z¹E¹.

D¹E² is an organic compound, wherein D¹ is an organic group comprising an unsaturated hydrocarbyl group having 2-12 carbon atoms, and E² is a reactive functional group comprising hydroxyl, amine, oxirane, or isocyanate. The organic group represented by D¹ typically comprises an unsaturated hydrocarbyl group having 2 to 12 carbon atoms, alternatively 2 to 11 carbon atoms, alternatively from 3 to 10 carbon atoms, alternatively from 3 to 6 carbon atoms, alternatively 3 or 4 carbon atoms, alternatively 3 carbon atoms. Examples of unsaturated hydrocarbyl groups represented by D¹ include, but are not limited to, alkenyl, such as vinyl, allyl, and butenyl; aralkenyl, eugenyl, styryl and cinnamyl; and alkynyl, such as ethynyl and propynyl.

The reactive functional groups represented by E² comprise hydroxyl, amine, oxirane, or isocyanate. The hydroxyl, amine, oxirane and isocyanate groups are as described above for E¹.

Examples of compounds represented by D¹E² include, but are not limited to, allyl alcohol, 3-buten-1-ol, 4-penten-1-ol, 5-hexen-1-ol, 6-septen-1-ol, 11-dodecen-1-ol, eugenol, 3-amino-1-propene, 4-amino-1-butene, 5-amino-1-pentene, 6-amino-1-hexene, 6-amino-1-cyclohexene, 12-amino-1-dodecene, 3,4-epoxy-1-butene, 1,2-epoxy-5-hexene, 1,2-epoxy-9-decene, allyl isocyanate, 1-isocyanato-3-butene, 1-isocyanato-4-pentene, 1-isocyanato-5-hexene, and phenylisocyanate. Compounds represented by D¹E² are available commercially.

The intermediate F¹ is formed by the reaction of Z¹E¹ and D¹E². Examples of the intermediate represented by F¹ include, but are not limited to, the following compounds:

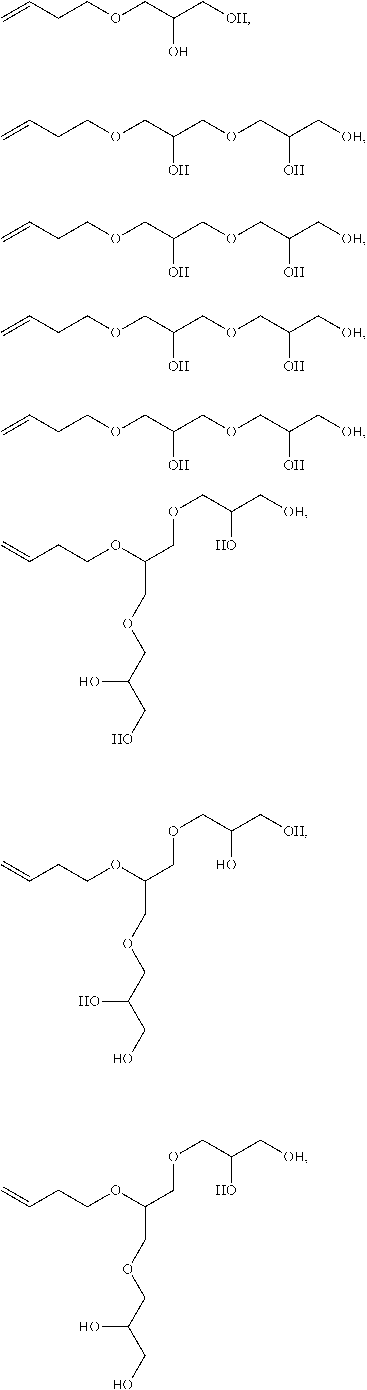

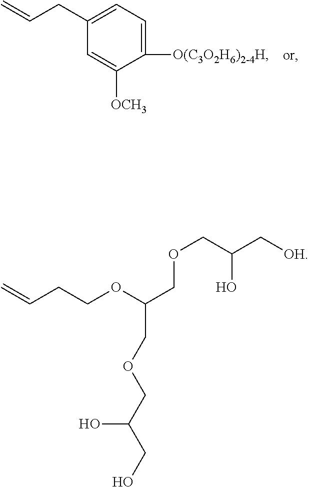

The intermediate F¹ is reacted with an organosilane of formula Si(OR¹)ₙ(R²)₃₋ₙH, where R¹ is an alkyl group containing 1 to 4 carbon atoms and R² is an alkyl group containing 1 to 4 carbon atoms, n is from 1 to 3, alternatively 2 or 3, alternatively 1, alternatively 2, alternatively 3, and a hydrosilylation catalyst.

The alkyl groups represented by R¹ typically have from 1 to 4 carbon atoms, alternatively 1 or 2 carbon atoms, alternatively 1 carbon atom, alternatively 2 carbon atoms. Examples of hydrocarbyl groups represented by R¹ include, but are not limited to, methyl, ethyl, propyl, and butyl. The alkyl groups represented by R² are as defined for R¹.

The hydrosilylation catalysts can be any catalyst known to catalyze a hydrosilylation reaction between any compound containing an SiH group and a compound comprising an unsaturated hydrocarbon such as alkene or alkyne group. In one embodiment, the hydrosilylation catalyst comprises platinum. Examples of catalysts include compounds such as ruthenium, rhodium, palladium, osmium, iridium or the like. Examples of platinum compounds that may be used as catalysts comprise chloroplatinic acid, platinum metal, a platinum metal-supported carrier such as platinum-supported alumina, platinum-supported silica, platinum-supported carbon black or the like. Platinum complexes such as platinum-vinylsiloxane complex, platinum phosphine complexes, platinum-phosphited complexes, platinum alcholate catalyst or the like may also be used. An effective amount of catalyst is used. As used herein “an effective amount of catalyst” is typically from 0.5 to 1,000 ppm as a platinum metal in the case of using a platinum catalysts.

Examples of organosilane compounds formed by Method A for preparing the organosilane include those of described above for formula (I).

A method for preparing an organosilane, the method comprising: reacting (a) an organosilane of formula Si(OR¹)ₙ(R²)₃₋ₙB¹, where R¹ is an alkyl group containing 1 to 4 carbon atoms and R² is an alkyl group containing 1 to 4 carbon atoms, n is from 1 to 3, and B¹ is an organic group comprising a reactive functional group, where the reactive functional group comprises hydroxyl, amine, oxirane, or isocyanate, and (b) an organic compound Z¹-E¹, wherein Z is a sugar, a monoglycerol group, a diglycerol, a polyglycerol, eugenol, or a xylitol group, E¹ is hydroxyl, amine or an organic group comprising a reactive functional group, wherein the reactive functional group comprises hydroxyl, amine, oxirane, or isocyanate, at a temperature and pressure sufficient to cause (a) and (b) to react.

The groups represented by R¹ and R² in the organosilane reacted in Method B for preparing the organosilane are as described above for Method A for preparing the organosilane.

Organic groups comprising a reactive group represented by B¹ include, but are not limited to hydrocarbyl groups having from 1 to 10 carbon atoms, alternatively, 1 to 7 carbon atoms, alternatively 1 to 3 carbon atoms, wherein the hydrocarbyl group is substituted with the reactive group. Examples of hydrocarbyl groups include eugenol, where the non-aromatic olefin (i.e., terminal unsaturation) is replaced by a terminal bond to the silicon atom of the organosilane, and alkyl, such as methyl, ethyl, propyl, butyl, hexyl, octyl, and decyl. The backbone of the hydrocarbyl group may be substituted with one or more of the following atoms and/or groups: oxygen, nitrogen, carbonyl, carboxyl, amide, and ureylene, alternatively, the backbone of the hydrocarbyl group is substituted with one or more of the following atoms and/or groups: oxygen, nitrogen, carbonyl, carboxyl, amide, and ureylene, alternatively oxygen, alternatively nitrogen.

The reactive group of the organic group B¹ is hydroxyl, amine, oxirane, or isocyanate. The reactive functional group is as described for Method A for preparing the organosilane above. B¹ may be the hydrosilylation reaction product of an organohydridosilane of formula Si(OR¹)ₙ(R²)₃₋ₙH with D¹E² above, wherein R¹, R², and n are as defined above.

The organic compound E¹Z¹ and the groups E¹ and Z¹ are as defined above for Method A for preparing the organosilane.

Examples of organosilane compounds formed by Method B for preparing the organosilane include those of described above for formula (I).

Method A and B for preparing an organosilane described above are conducted at a temperature sufficient to cause the reactions to take place. A temperature sufficient to cause the reaction to place is typically from 25° C. to 300° C., alternatively from 35° C. to 150° C., alternatively from 50° C. to 60° C., alternatively from 45° C. to 100° C.

Method A and B for preparing an organosilane described above are conducted at a pressure sufficient for the reaction to take place. A pressure sufficient for the reaction to take place typically means a pressure from atmospheric pressure to a pressure above atmospheric pressure, alternatively at atmospheric pressure, alternatively at a pressure above atmospheric pressure, alternatively at a pressure from 0 to 100 kPa gauge pressure, alternatively at a pressure from 10 kPa to 100 kPa.

Method A and B for preparing an organosilane described above are conducted for a time sufficient for the reaction to take place. One skilled in the art will understand that the time sufficient for the reaction to take place will vary with the temperature and the pressure of the reaction. A time sufficient for the reaction is typically at least 10 minutes, alternatively from 30 minutes to 20 hours, alternatively from 2 to 10 hours.

Method A and B for preparing an organosilane described above may be conducted in any reactor typically used for chemical reactions at elevated temperate such as a three neck glass flask, a column, sealed tube, film, such as a thin film or falling film reactor. One skilled in the art would know how to select an appropriate reactor to conduct the method for preparing the organosilane.

The organosilanes of formula (I) described above and produced by method of preparing an organosilane A or Method B for preparing the organosilane can be used in many applications and provide benefits including, but not limited to, improved dispersibility of powders, transmittance, antifog and antifouling coatings.

The composition is a personal care composition, surface treating composition, an antifog composition, a coating composition, a surface treated powder, a paint composition, or an ink composition.

The invention also provides that the composition is a personal care composition, which may also be described as personal care products or compositions. The personal care compositions include the organosilane described above. The personal care compositions may be in the form of a cream, a gel, a powder, a paste, or a freely pourable liquid. Generally, such compositions can generally be prepared at room temperature if no solid materials at room temperature are present in the compositions, using simple propeller mixers, Brookfield counter-rotating mixers, or homogenizing mixers. No special equipment or processing conditions are typically required. Depending on the type of form made, the method of preparation will be different, but such methods are well known in the art.

The personal care compositions may be functional with respect to the portion of the body to which it is applied, cosmetic, therapeutic, or some combination thereof. Conventional examples of such products include, but are not limited to, antiperspirants and deodorants, skin care creams, skin care lotions, moisturizers, facial treatments such as acne or wrinkle removers, personal and facial cleansers, bath oils, perfumes, colognes, sachets, sunscreens, pre-shave and after-shave lotions, shaving soaps, and shaving lathers, hair shampoos, hair conditioners, hair colorants, hair relaxants, hair sprays, mousses, gels, permanents, depilatories, and cuticle coats, make-ups, color cosmetics, foundations, concealers, blushes, lipsticks, eyeliners, mascara, oil removers, color cosmetic removers, and powders, medicament creams, pastes or sprays including anti-acne, dental hygienic, antibiotic, healing promotive, nutritive and the like, which may be preventative and/or therapeutic. In general, the personal care compositions may be formulated with a carrier that permits application in any conventional form, including but not limited to, liquids, rinses, lotions, creams, pastes, gels, foams, mousses, ointments, sprays, aerosols, soaps, sticks, soft solids, solid gels, and gels. Suitable carriers are appreciated in the art.

The personal care composition can be used in or for a variety of personal, household, and healthcare applications. In particular, personal care compositions of the present disclosure may be used in the personal care products as described in U.S. Pat. Nos. 6,051,216; 5,919,441; and 5,981,680; WO 2004/060271 and WO 2004/060101; in sunscreen compositions as described in WO 2004/060276; in cosmetic compositions also containing film-forming resins, as described in WO 03/105801; in the cosmetic compositions as described in US Pat. App. Pub. Nos. 2003/0235553; 2003/0072730 and 2003/0170188, in EP Pat. Nos. 1,266,647; 1,266,648 and 1,266,653, in WO 03/105789, WO 2004/000247 and WO 03/106614; as additional agents to those described in WO 2004/054523; in long wearing cosmetic compositions as described in US Pat. App. Pub. No. 2004/0180032; and/or in transparent or translucent care and/or make up compositions as described in WO 2004/054524, all of which are expressly incorporated herein by reference in various non-limiting embodiments.

The personal care composition can be used by standard methods, such as applying them to the human body, for example, skin or hair, using applicators, brushes, applying by hand, pouring them and/or possibly rubbing or massaging the composition onto or into the body. Removal methods, for example for color cosmetics are also well known standard methods, including washing, wiping, peeling and the like. For use on the skin, the personal care composition may be used in a conventional manner for example for conditioning the skin. An effective amount of the personal care composition may be applied to the skin. Such effective amounts generally be from 1 mg/cm² to 3 mg/cm². Application to the skin typically includes working the personal care composition into the skin. This method for applying to the skin typically includes the steps of contacting the skin with the personal care composition in an effective amount and then rubbing the personal care composition into the skin. These steps can be repeated as many times as desired to achieve the desired benefit.

Use of the personal care compositions on hair may use a conventional manner for conditioning hair. An effective amount of the personal care composition for conditioning hair is applied to the hair. Such effective amounts generally range from 1 g to 50 g, typically from 1 g to 20 g. Application to the hair typically includes working the personal care composition through the hair such that most or all of the hair is contacted with the personal care composition. This method for conditioning the hair typically includes the steps of applying an effective amount of the personal care composition to the hair, and then working the personal care composition through the hair. These steps can be repeated as many times as desired to achieve the desired conditioning benefit.

Non-limiting examples of additives which may be formulated into the personal care composition include, but are not limited to, silicones, anti-oxidants, cleansing agents, colorants, conditioning agents, deposition agents, electrolytes, emollients and oils, exfoliating agents, foam boosting agents, fragrances, humectants, occlusive agents, pediculicides, pH control agents, pigments, preservatives, biocides, solvents, stabilizers, sun-screening agents, suspending agents, tanning agents, other surfactants, thickeners, vitamins, botanicals, waxes, rheology-modifying agents, anti-dandruff, anti-acne, anti-carie and wound healing-promotion agents.

The personal care composition, such as a shampoo or cleanser, may include at least one anionic detersive surfactant. This can be any of the well-known anionic detersive surfactants typically used in shampoo formulations. These anionic detersive surfactants can function as cleansing agents and foaming agents in the shampoo compositions. The anionic detersive surfactants are exemplified by alkali metal sulforicinates, sulfonated glyceryl esters of fatty acids such as sulfonated monoglycerides of coconut oil acids, salts of sulfonated monovalent alcohol esters such as sodium oleylisethianate, amides of amino sulfonic acids such as the sodium salt of oleyl methyl tauride, sulfonated products of fatty acids nitriles such as palmitonitrile sulfonate, sulfonated aromatic hydrocarbons such as sodium alpha-naphthalene monosulfonate, condensation products of naphthalene sulfonic acids with formaldehyde, sodium octahydroanthracene sulfonate, alkali metal alkyl sulfates such as sodium lauryl sulfate, ammonium lauryl sulfate or triethanol amine lauryl sulfate, ether sulfates having alkyl groups of 8 or more carbon atoms such as sodium lauryl ether sulfate, ammonium lauryl ether sulfate, sodium alkyl aryl ether sulfates, and ammonium alkyl aryl ether sulfates, alkylarylsulfonates having 1 or more alkyl groups of 8 or more carbon atoms, alkylbenzenesulfonic acid alkali metal salts exemplified by hexylbenzenesulfonic acid sodium salt, octylbenzenesulfonic acid sodium salt, decylbenzenesulfonic acid sodium salt, dodecylbenzenesulfonic acid sodium salt, cetylbenzenesulfonic acid sodium salt, and myristylbenzenesulfonic acid sodium salt, sulfuric esters of polyoxyethylene alkyl ether including CH₃(CH₂)₆CH₂O(C₂H₄O)₂SO₃H, CH₃(CH₂)₇CH₂O(C₂H₄O)_{3.5}SO₃H, CH₃(CH₂)₈CH₂O(C₂H4O)₈SO₃H, CH₃(CH₂)₁₉CH₂O(C₂H₄O)₄SO₃H, and CH₃(CH₂)₁₀CH₂O(C₂H₄O)₆SO₃H, sodium salts, potassium salts, and amine salts of alkylnaphthylsulfonic acid. Typically, the detersive surfactant is chosen from sodium lauryl sulfate, ammonium lauryl sulfate, triethanolamine lauryl sulfate, sodium lauryl ether sulfate, and ammonium lauryl ether sulfate. The anionic detersive surfactant can be present in the shampoo composition in an amount from 5 to 50 wt % and typically 5 to 25 wt % based on the total weight of the shampoo composition.

The personal care composition may include at least one amphoteric surfactant. Amphoteric surfactants are known in the art and available commercially. The amphoteric surfactant is typically present at levels from 0.001 to 50% (w/w), alternatively from 5 to 25% (w/w), based on the weight of the personal care formulation. As examples of amphoteric surfactants, mention may be made of imidazoline-type, amidobetaine-type, alkylbetaine-type, alkylamidobetaine-type, alkylsulfobetaine-type, am idosulfobetaine-type, hydroxysulfobetaine-type, carbobetaine-type, phosphobetaine-type, aminocarboxylic acid-type, and amidoamino acid-type amphoteric surfactants. More particularly, as examples thereof, mention may be made of imidazoline-type amphoteric surfactants such as sodium 2-undecyl-N,N,N-(hydroxyethylcarboxymethyl)-2-imidazoline, 2-cocoyl-2-imidazolinium hydroxide-1-carboxyethyloxy disodium salt and the like; alkylbetaine-type amphoteric surfactants such as lauryl dimethylaminoacetic acid betaine, myristyl betaine and the like; and amidobetaine-type amphoteric surfactants such as coconut oil fatty acid amidopropyl dimethylamino acetic acid betaine, palm kernel oil fatty acid amidopropyl dimethylamino acetic acid betaine, beef tallow fatty acid amidopropyl dimethylamino acetic acid betaine, hardened beef tallow fatty acid amidopropyl dimethylamino acetic acid betaine, lauric amidopropyl dimethylamino acetic acid betaine, myristic amidopropyl-dimethylamino acetic acid betaine, palmitic amidopropyl-dimethylamino acetic acid betaine, stearic amidopropyl dimethylamino acetic acid betaine, oleic amidopropyl dimethylamino acetic acid betaine and the like; alkyl sulfobetaine-type amphoteric surfactants such as coconut oil fatty acid dimethyl sulfopropyl betaine and the like; alkylhydroxy sulfobetaine-type amphoteric surfactants such as lauryl dimethylaminohydroxy sulfobetaine and the like; phosphobetaine-type amphoteric surfactants such as laurylhydroxy phosphobetaine and the like; amidoamino acid-type amphoteric surfactants such as sodium N-lauroyl-N′-hydroxyethyl-N′-carboxymethyl ethylenediamine, sodium N-oleoyl-N′-hydroxyethyl-N′-carboxymethyl ethylenediamine, sodium N-cocoyl-N′-hydroxyethyl-N′-carboxymethyl ethylenediamine, potassium N-lauroyl-N′-hydroxyethyl-N′-carboxymethyl ethylenediamine, potassium N-oleoyl-N′-hydroxyethyl-N′-carboxymethyl ethylenediamine, sodium N-lauroyl-N-hydroxyethyl-N′-carboxymethyl ethylenediamine, sodium N-oleoyl-N-hydroxyethyl-N′-carboxymethyl ethylenediamine, sodium N-cocoyl-N-hydroxyethyl-N′-carboxymethyl ethylenediamine, monosodium N-lauroyl-N-hydroxyethyl-N′,N′-dicarboxymethyl ethylenediamine, monosodium N-oleoyl-N-hydroxyethyl-N′,N′-dicarboxymethyl ethylenediamine, monosodium N-cocoyl-N-hydroxyethyl-N′,N′-dicarboxymethyl ethylenediamine, disodium N-lauroyl-N-hydroxyethyl-N′,N′-dicarboxymethyl ethylenediamine, disodium N-oleoyl-N-hydroxyethyl-N′,N′-dicarboxymethyl ethylenediamine, disodium N-cocoyl-N-hydroxyethyl-N′,N′-dicarboxymethyl ethylenediamine and the like.

The personal care composition may include at least one cationic deposition aid, typically a cationic deposition polymer. The cationic deposition aid is typically present at levels of from 0.001 to 5%, typically from 0.01 to 1%, more typically from 0.02% to 0.5% by weight. The cationic deposition polymer may be a homopolymer or be formed from two or more types of monomers. The molecular weight of the cationic deposition polymer is typically from 5,000 to 10,000,000, typically at least 10,000 and typically from 100,000 to 2,000,000. The cationic deposition polymers typically have cationic nitrogen containing groups such as quaternary ammonium or protonated amino groups, or a combination thereof. The cationic charge density has been found to need to be at least 0.1 meq/g, typically above 0.8 or higher. The cationic charge density should not exceed 4 meq/g, it is typically less than 3 and more typically less than 2 meq/g. The charge density can be measured using the Kjeldahl method and is within the above limits at the desired pH of use, which will in general be from 3 to 9 and typically from 4 to 8. It is contemplated that any and all values or ranges of values between those described above may also be utilized. The cationic nitrogen-containing group is typically present as a substituent on a fraction of the total monomer units of the cationic deposition polymer. Thus when the cationic deposition polymer is not a homopolymer it can include spacer noncationic monomer units. Such cationic deposition polymers are described in the CTFA Cosmetic Ingredient Directory, 3rd edition, which is expressly incorporated herein by reference in one or more non-limiting embodiments. Suitable cationic deposition aids include, for example, copolymers of vinyl monomers having cationic amine or quaternary ammonium functionalities with water soluble spacer monomers such as (meth)acrylamide, alkyl and dialkyl (meth)acrylamides, alkyl (meth)acrylate, vinyl caprolactone and vinyl pyrrolidine. The alkyl and dialkyl substituted monomers typically have C1-C7 alkyl groups, more typically C1-C3 alkyl groups. Other suitable spacers include vinyl esters, vinyl alcohol, maleic anhydride, propylene glycol and ethylene glycol.

The cationic amines can be primary, secondary or tertiary amines, depending upon the particular species and the pH of the composition. In general secondary and tertiary amines, especially tertiary, are typical. Amine substituted vinyl monomers and amines can be polymerized in the amine form and then converted to ammonium by quaternization. Suitable cationic amino and quaternary ammonium monomers include, for example, vinyl compounds substituted with dialkyl aminoalkyl acrylate, dialkylamino alkylmethacrylate, monoalkylaminoalkyl acrylate, monoalkylaminoalkyl methacrylate, trialkyl methacryloxyalkyl ammonium salt, trialkyl acryloxyalkyl ammonium salt, diallyl quaternary ammonium salts, and vinyl quaternary ammonium monomers having cyclic cationic nitrogen-containing rings such as pyridinium, imidazolium, and quaternized pyrrolidine, e.g. alkyl vinyl imidazolium, and quaternized pyrrolidine, e.g. alkyl vinyl imidazolium, alkyl vinyl pyridinium, alkyl vinyl pyrrolidine salts. The alkyl portions of these monomers are typically lower alkyls such as the C1-C7 alkyls, more typically C1 and C2 alkyls. Suitable amine-substituted vinyl monomers for use herein include dialkylaminoalkyl acrylate, dialkylaminoalkyl methacrylate, dialkylaminoalkyl acrylamide, and dialkylaminoalkyl methacrylamide, wherein the alkyl groups are typically C1-C7 hydrocarbyls, more typically C1-C3, alkyls. The cationic deposition aids can include combinations of monomer units derived from amine- and/or quaternary ammonium-substituted monomer and/or compatible spacer monomers. Suitable cationic deposition aids include, for example: copolymers of 1-vinyl-2-pyrrolidine and 1-vinyl-3-methylimidazolium salt (e.g. Chloride salt) (referred to in the industry by the Cosmetic, Toiletry, and Fragrance Association, “CTFA” as Polyquaternium-16) such as those commercially available from BASF Wyandotte Corp. (Parsippany, N.J., USA) under the LUVIQUAT tradename (e.g. LUVIQUAT FC 370); copolymers of 1-vinyl-2-pyrrolidine and dimethylaminoethyl methacrylate (referred to in the industry by CTFA as Polyquaternium-11) such as those commercially from Gar Corporation (Wayne, N.J., USA) under the GAFQUAT tradename (e.g. GAFQUAT 755N); cationic diallyl quaternary ammonium-containing polymer including, for example, dimethyl diallyammonium chloride homopolymer and copolymers of acrylamide and dimethyl diallyammonium chloride, referred to in the industry (CTFA) as Polyquaternium 6 and Polyquaternium 7, respectively; mineral acid salts of aminoalkyl esters of homo- and co-polymers of unsaturated carboxylic acids having from 3 to 5 carbon atoms, as described in U.S. Pat. No. 4,009,256; and cationic polyacrylamides as described in UK Application No. 9403156.4 (WO95/22311), each of which is expressly incorporated herein in one or more non-limiting embodiments.

Other cationic deposition aids that can be used include polysaccharide polymers, such as cationic cellulose derivatives and cationic starch derivatives. Cationic polysaccharide polymer materials suitable for use in compositions of the disclosure include those of the formula: A-O(R—N⁺R¹R²R³X⁻) wherein A is an anhydroglucose residual group, such as starch or cellulose anhydroglucose residual, R is an alkylene oxyalklene, polyoxyalkylene, or hydroxyalkylene group, or combination thereof, R¹, R² and R³ independently are alkyl, aryl, alkylaryl, arylalkyl, alkoxyalkyl, or alkoxyaryl groups, each group containing up to 18 carbon atoms, and the total number of carbon atoms for each cationic moiety (i.e., the sum of carbon atoms in R¹, R², R³) typically being 20 or less, and X is an anionic counterion, as previously described. Cationic cellulose is available from Amerchol Corp. (Edison, N.J., USA) in their Polymer iR (trade mark) and LR (trade mark) series of polymers, as salts of hydroxyethyl cellulose reacted with trimethyl ammonium substituted epoxide, referred to in the industry (CTFA) as Polyquaternium 10.

Another type of cationic cellulose includes the polymeric quaternary ammonium salts of hydroxyethyl cellulose reacted with lauryl dimethyl ammonium-substituted epoxide, referred to in the industry (CTFA) as Polyquaternium 24. These materials are available from Amerchol Corp. (Edison, N.J., USA) under the tradename Polymer LM-200. Other cationic deposition aids that can be used include cationic guar gum derivatives, such as guar hydroxypropyltrimonium chloride (Commercially available from Celanese Corp. in their Jaguar trademark series). Other materials include quaternary nitrogen-containing cellulose ethers (e.g. as described in U.S. Pat. No. 3,962,418), and copolymers of etherified cellulose and starch (e.g. as described in U.S. Pat. No. 3,958,581), each of which is expressly incorporated herein by reference in one or more non-limiting embodiments.

The personal care composition may include a foam boosting agent. A foam boosting agent is an agent which increases the amount of foam available from a system at a constant molar concentration of surfactant, in contrast to a foam stabilizer which delays the collapse of a foam. Foam building is provided by adding to the aqueous media, a foam boosting effective amount of a foam boosting agent. The foam boosting agent is typically chosen from fatty acid alkanolamides and amine oxides. The fatty acid alkanolamides are exemplified by isostearic acid diethanolamide, lauric acid diethanolamide, capric acid diethanolamide, coconut fatty acid diethanolamide, linoleic acid diethanolamide, myristic acid diethanolamide, oleic acid diethanolamide, stearic acid diethanolamide, coconut fatty acid monoethanolamide, oleic acid monoisopropanolamide, and lauric acid monoisopropanolamide. The amine oxides are exemplified by N-cocodimethylamine oxide, N-lauryl dimethylamine oxide, N-myristyl dimethylamine oxide, N-stearyl dimethylamine oxide, N-cocamidopropyl dimethylamine oxide, N-tallowamidopropyl dimethylamine oxide, bis(2-hydroxyethyl) C12-15 alkoxypropylamine oxide.

Typically a foam boosting agent is chosen from lauric acid diethanolamide, N-lauryl dimethylamine oxide, coconut acid diethanolamide, myristic acid diethanolamide, and oleic acid diethanolamide. The foam boosting agent is typically present in the shampoo compositions in an amount from 1 to 15 wt % and more typically 2 to 10 wt % based on the total weight of the composition. The composition may further include a polyalkylene glycol to improve lather performance. Concentration of the polyalkylene glycol in the shampoo composition may be from 0.01% to 5%, typically from 0.05% to 3%, and more typically from 0.1% to 2%, by weight of the shampoo composition. The optional polyalkylene glycols are characterized by the general formula: H(OCH₂CHR)ₙ—OH wherein R is chosen from H, methyl, and combinations thereof. When R is H, these materials are polymers of ethylene oxide, which are also known as polyethylene oxides, polyoxyethylenes, and polyethylene glycols. When R is methyl, these materials are polymers of propylene oxide, which are also known as polypropylene oxides, polyoxypropylenes, and polypropylene glycols. When R is methyl, it is also understood that various positional isomers of the resulting polymers can exist. In the above structure, n has an average value of from 1500 to 25,000, typically from 2500 to 20,000, and more typically from 3500 to 15,000. Polyethylene glycol polymers useful herein are PEG-2M wherein R equals H and n has an average value of 2,000 (PEG-2M is also known as Polyox WSR9N-10, which is available from Union Carbide and as PEG-2,000); PEG-5M wherein R equals H and n has an average value of 5,000 (PEG-5M is also known as Polyox WSRO N-35 and Polyox WSRS N-80, both available from Union Carbide and as PEG-5,000 and Polyethylene Glycol 300,000); PEG-7M wherein R equals H and n has an average value of 7,000 (PEG-7M is also known as Polyox WSRO N-750 available from Union Carbide); PEG-9M wherein R equals H and n has an average value of 9,000 (PEG 9-M is also known as Polyox WSRS N-3333 available from Union Carbide); and PEG-14 M wherein R equals H and n has an average value of 14,000 (PEG-14M is also known as Polyox WSRO N-3000 available from Union Carbide). Other useful polymers include the polypropylene glycols and mixed polyethylene/polypropylene glycols.

The personal care composition may include a suspending agent at concentrations effective for suspending a silicone conditioning agent, or other water-insoluble material, in dispersed form in the personal care composition. Such concentrations may be from 0.1% to 10%, typically from 0.3% to 5.0%, by weight of the personal care composition. Suspending agents include crystalline suspending agents which can be categorized as acyl derivatives, long chain amine oxides, and combinations thereof, concentrations of which can be from 0.1% to 5.0%, typically from 0.5% to 3.0%, by weight of the shampoo compositions. These suspending agents are described in U.S. Pat. No. 4,741,855, which is expressly incorporated herein by reference in one or more non-limiting embodiments. These typical suspending agents include ethylene glycol esters of fatty acids typically having from 16 to 22 carbon atoms. More typical are the ethylene glycol stearates, both mono and distearate, but particularly the distearate containing less than 7% of the mono stearate.

Other suitable suspending agents include alkanol amides of fatty acids, typically having from 16 to 22 carbon atoms, more typically 16 to 18 carbon atoms, typical examples of which include stearic monoethanolamide, stearic diethanolamide, stearic monoisopropanolamide and stearic monoethanolamide stearate. Other long chain acyl derivatives include long chain esters of long chain fatty acids (e.g. stearyl stearate, cetyl palmitate, etc.); glyceryl esters (e.g. glyceryl distearate) and long chain esters of long chain alkanol amides (e.g. stearamide diethanolamide distearate, stearamide monoethanolamide stearate). Long chain acyl derivatives, ethylene glycol esters of long chain carboxylic acids, long chain amine oxides, and alkanol amides of long chain carboxylic acids in addition to the typical materials listed above may be used as suspending agents. For example, it is contemplated that suspending agents with long chain hydrocarbyls having C8-C22 chains may be used. Other long chain acyl derivatives suitable for use as suspending agents include N,N-dihydrocarbyl amido benzoic acid and soluble salts thereof (e.g. Na, K), particularly N,N-di(hydrogenated) C16, C18 and tallow amido benzoic acid species of this family, which are commercially available from Stepan Company (Northfield, Ill., USA). Examples of suitable long chain amine oxides for use as suspending agents include alkyl (C16-C22) dimethyl amine oxides, e.g. stearyl dimethyl amine oxide. Other suitable suspending agents include xanthan gum at concentrations ranging from 0.3% to 3%, typically from 0.4% to 1.2%, by weight of the shampoo compositions. The use of xanthan gum as a suspending agent is described, for example, in U.S. Pat. No. 4,788,006, which is expressly incorporated herein by reference in one or more non-limiting embodiments. Combinations of long chain acyl derivatives and xanthan gum may also be used as a suspending agent in the shampoo compositions. Such combinations are described in U.S. Pat. No. 4,704,272, which is expressly incorporated herein by reference in one or more non-limiting embodiments. Other suitable suspending agents include carboxyvinyl polymers. Typical among these polymers are the copolymers of acrylic acid crosslinked with polyallylsucrose as described in U.S. Pat. No. 2,798,053, which is expressly incorporated herein by reference in one or more non-limiting embodiments. Examples of these polymers include Carbopol 934, 940, 941, and 956, available from B.F. Goodrich Company. Other suitable suspending agents include primary amines having a fatty alkyl moiety having at least 16 carbon atoms, examples of which include palmitamine or stearamine, and secondary amines having two fatty alkyl moieties each having at least 12 carbon atoms, examples of which include dipalmitoylamine or di(hydrogenated tallow)amine. Still other suitable suspending agents include di(hydrogenated tallow)phthalic acid amide, and crosslinked maleic anhydride-methyl vinyl ether copolymer. Other suitable suspending agents may be used in the shampoo compositions, including those that can impart a gel-like viscosity to the composition, such as water soluble or colloidally water soluble polymers like cellulose ethers (e.g. methylcellulose, hydroxybutyl methylcellulose, hyroxypropylcellulose, hydroxypropyl methylcellulose, hydroxyethyl ethylcellulose and hydroxyethylcellulose), guar gum, polyvinyl alcohol, polyvinyl pyrrolidone, hydroxypropyl guar gum, starch and starch derivatives, and other thickeners, viscosity modifiers, gelling agents, etc.

The personal care compositions may include one or more water-soluble materials. Examples of water-soluble materials include, but are not limited to, water-soluble emollients, polyhydric alcohols, or lower monovalent alcohols. Water-soluble emollients include, but are not limited to, lower molecular weight aliphatic diols such as propylene glycol and butylene glycol; polyols such as glycerine and sorbitol; and polyoxyethylene polymers such as polyethylene glycol 200. As the alcohols, one type or two or more types of polyhydric alcohols and/or lower monovalent alcohols can be used. As examples of lower alcohols, mention may be made of ethanol, isopropanol, n-propanol, t-butanol, s-butanol and the like. As examples of polyhydric alcohols, mention may be made of divalent alcohols such as 1,3-propanediol, 1,3-butylene glycol, 1,2-butylene glycol, propylene glycol, trimethylene glycol, tetramethylene glycol, 2,3-butylene glycol, pentamethylene glycol, 2-butene-1,4-diol, dibutylene glycol, pentyl glycol, hexylene glycol, octylene glycol and the like; trivalent alcohols such as glycerol, trimethylolpropane, 1,2,6-hexanetriol and the like; polyhydric alcohols having tetra- or more valences such as pentaerythritol, xylitol and the like; sugar alcohols such as sorbitol, mannitol, maltitol, maltotriose, sucrose, erytritol, glucose, fructose, starch-decomposed products, maltose, xylitose, starch-decomposed reduction alcohols and the like. In addition to the aforementioned polyhydric alcohols having a low molecular weight, mention may be made of polyhydric alcohol polymers such as diethylene glycol, dipropylene glycol, triethylene glycol, polypropylene glycol, tetraethylene glycol, diglycerol, polyethylene glycol, triglycerol, tetraglycerol, polyglycerol and the like. Among these, 1,3-propanediol, 1,3-butylene glycol, sorbitol, dipropylene glycol, glycerol, and polyethylene glycol are, in particular, preferred. The blending amount of the water-soluble material may preferably range from 0.1 to 50% by weight (mass) with respect to the total amount of the cosmetic. The water-soluble materials can be blended in order to improve storage stability of the cosmetic or personal care composition, in an amount ranging from about 5 to 30% by weight (mass), with respect to the total amount of the cosmetic or personal care composition. The specific type and amount of water soluble emollient(s), polyhydric alcohol, and monovalent alcohol employed will vary depending on the desired aesthetic characteristics of the composition, and is readily determined by one skilled in the art. This is one of the preferable modes for carrying out the present invention.

The personal care compositions may include one or more oils independent from the carrier fluid described above. The term “oil” as used herein describes any material which is substantially insoluble in water. Suitable oils include, but are not limited to, natural oils such as coconut oil; hydrocarbons such as mineral oil and hydrogenated polyisobutene; fatty alcohols such as octyldodecanol; esters such as C12-C15 alkyl benzoate; diesters such as propylene dipelarganate; and triesters, such as glyceryl trioctanoate and silicones especially cyclomethicone and dimethicone and combinations thereof. Suitable low viscosity oils have a viscosity of 5 to 100 mPas at 25° C., and are generally esters having the structure RCO—OR′ wherein RCO represents the carboxylic acid radical and wherein OR′ is an alcohol residue. Examples of these low viscosity oils include isotridecyl isononanoate, PEG-4 diheptanoate, isostearyl neopentanoate, tridecyl neopentanoate, cetyl octanoate, cetyl palmitate, cetyl ricinoleate, cetyl stearate, cetyl myristate, coco-dicaprylate/caprate, decyl isostearate, isodecyl oleate, isodecyl neopentanoate, isohexyl neopentanoate, octyl palmitate, dioctyl malate, tridecyl octanoate, myristyl myristate, octododecanol, or combinations of octyldodecanol, acetylated lanolin alcohol, cetyl acetate, isododecanol, polyglyceryl-3-diisostearate, or combinations thereof. The high viscosity surface oils generally have a viscosity of 200-1,000,000 mPas at 25° C., typically a viscosity of 100,000-250,000 mPas. Surface oils include castor oil, lanolin and lanolin derivatives, triisocetyl citrate, sorbitan sesquioleate, 010-18 triglycerides, caprylic/capric/triglycerides, coconut oil, corn oil, cottonseed oil, glyceryl triacetyl hydroxystearate, glyceryl triacetyl ricinoleate, glyceryl trioctanoate, hydrogenated castor oil, linseed oil, mink oil, olive oil, palm oil, illipe butter, rapeseed oil, soybean oil, sunflower seed oil, tallow, tricaprin, trihydroxystearin, triisostearin, trilaurin, trilinolein, trimyristin, triolein, tripalmitin, tristearin, walnut oil, wheat germ oil, cholesterol, or combinations thereof. The suggested ratio of low viscosity to high viscosity oils in the oil phase is 1:15 to 15:1, typically 1:10 to 10:1 respectively. The typical formulation of the disclosure includes 1 to 20% of a combination of low viscosity and high viscosity surface oils.

Mineral oils, such as liquid paraffin or liquid petroleum, or animal oils, such as perhydrosqualene or arara oil, or alternatively of vegetable oils, such as sweet almond, calophyllum, palm, castor, avocado, jojaba, olive or cereal germ oil, may be utilized. It is also possible to use esters of lanolic acid, of oleic acid, of lauric acid, of stearic acid or of myristic acid, for example; alcohols, such as oleyl alcohol, linoleyl or linolenyl alcohol, isostearyl alcohol or octyldodecanol; or acetylglycerides, octanoates, decanoates or ricinoleates of alcohols or of polyalcohols. It is alternatively possible to use hydrogenated oils which are solid at 25° C., such as hydrogenated castor, palm or coconut oils, or hydrogenated tallow; mono-, di-, tri- or sucroglycerides; lanolins; or fatty esters which are solid at 25° C.

The personal care composition may include a cosmetic ingredient. A cosmetic ingredient is an ingredient that imparts a cosmetic effect to the hair, skin, or nails. A cosmetic effect is a change to the feel or appearance of the skin, hair, or nails. One skilled in the art would understand what a cosmetic ingredient is.

The personal care compositions may include various waxes. The waxes generally have a melting point of from 35 to 120° C. at atmospheric pressure. Waxes in this category include synthetic wax, ceresin, paraffin, ozokerite, illipe butter, beeswax, carnauba, microcrystalline, lanolin, lanolin derivatives, candelilla, cocoa butter, shellac wax, spermaceti, bran wax, capok wax, sugar cane wax, montan wax, whale wax, bayberry wax, or combinations thereof. In one embodiment, the personal care composition includes 10-30% of a combination of waxes. Mention may be made, among the waxes capable of being used as non-silicone fatty substances, of animal waxes, such as beeswax; vegetable waxes, such as carnauba, candelilla, ouricury or japan wax or cork fibre or sugarcane waxes; mineral waxes, for example paraffin or lignite wax or microcrystalline waxes or ozokerites; synthetic waxes, including polyethylene waxes, and waxes obtained by the Fischer-Tropsch synthesis. Mention may be made, among the silicone waxes, of polymethylsiloxane alkyls, alkoxys and/or esters.

The personal care compositions may include a powder. The powder may be generally defined as dry, particulate matter having a particle size of 0.02-50 microns. The powder may be colored or non-colored (for example white). Suitable powders include bismuth oxychloride, titanated mica, fumed silica, spherical silica beads, polymethylmethacrylate beads, micronized teflon, boron nitride, acrylate polymers, aluminum silicate, aluminum starch octenylsuccinate, bentonite, calcium silicate, cellulose, chalk, corn starch, diatomaceous earth, fuller's earth, glyceryl starch, hectorite, hydrated silica, kaolin, magnesium aluminum silicate, magnesium carbonate, magnesium hydroxide, magnesium oxide, magnesium silicate, magnesium trisilicate, maltodextrin, montmorillonite, microcrystalline cellulose, rice starch, silica, talc, mica, titanium dioxide, zinc laurate, zinc myristate, zinc neodecanoate, zinc rosinate, zinc stearate, polyethylene, alumina, attapulgite, calcium carbonate, calcium silicate, dextran, kaolin, nylon, silica silylate, silk powder, serecite, soy flour, tin oxide, titanium hydroxide, trimagnesium phosphate, walnut shell powder, or combinations thereof. The powder may be surface treated with the organosilane described above, lecithin, amino acids, mineral oil, silicone oil, or various other agents either alone or in combination, which coat the powder surface and render the particles hydrophobic or hydrophilic in nature. In one embodiment, the powder is treated with the organosilane, alternatively the powder is treated with the organosilane then incorporated into the personal care formulation, alternatively zinc oxide or titanium dioxide are surface treated with the organosilane then incorporated into the personal care formulation. The surface treated powder may be incorporated into the personal care formulation using techniques known in the art and described below.

The powder may also include or be an organic and/or inorganic pigment. Organic pigments are generally various aromatic types including azo, indigoid, triphenylmethane, anthraquinone, and xanthine dyes which are designated as D&C and FD&C blues, browns, greens, oranges, reds, yellows, etc. Inorganic pigments generally consist of insoluble metallic salts of certified color additives, referred to as the Lakes or iron oxides. A pulverulent coloring agent, such as carbon black, chromium or iron oxides, ultramarines, manganese pyrophosphate, iron blue, and titanium dioxide, pearlescent agents, generally used as a combination with colored pigments, or some organic dyes, generally used as a combination with colored pigments and commonly used in the cosmetics industry, can be added to the composition. In general, these coloring agents can be present in an amount by weight from 0 to 20% with respect to the weight of the personal care composition.

The powder may comprise a silicone powder. Silicone powders are compositions having a organopolysiloxane in powder form, that is, in which water or liquid or solvent has been removed. The silicone powder can be prepared from an emulsion of a silicone (organopolysiloxane) by removing the water or any solvent. The silicone or organopolysiloxane can be a silicone elastomer, a silicone resin, a silicone gum or a silicone fluid.

As the powders, silicone elastomer powders can be used. The silicone elastomer powder is a crosslinked product of a linear diorganopolysiloxane mainly formed from a diorganosiloxane unit (D unit). The silicone elastomer powder can be preferably produced by crosslink-reacting an organohydrogenpolysiloxane having a silicon-binding hydrogen atom at the side chain or the terminal and a diorganopolysiloxane having an unsaturated hydrocarbon group such as an alkenyl group or the like at the side chain or the terminal, in the presence of a catalyst for a hydrosilylation reaction. The silicone elastomer powder has an increased flexibility and elasticity, and exhibits a superior oil-absorbing property, as compared with a silicone resin powder formed from T units and Q units. For this reason, the silicone elastomer powder absorbs sebum on the skin and can prevent makeup running. In addition, when a surface treatment is carried out by the aforementioned sugar alcohol-modified organopolysiloxane, a moisturized feeling on touch can be imparted without reducing a suede-like feeling on touch of the silicone elastomer powder. In addition, in the case of blending the aforementioned sugar alcohol-modified organopolysiloxane together with the silicone elastomer powder in a cosmetic, dispersion stability of the aforementioned powder in the entire cosmetic can be improved, and a stable cosmetic over time can be obtained.

The silicone elastomer powders can be in various forms such as a spherical form, a flat form, an amorphous form and the like. The silicone elastomer powders may be in the form of an oil dispersant. In the cosmetic of the present invention, silicone elastomer powders in the form of particles, which have a primary particle size observed by an electron microscope and/or an average primary particle size measured by a laser diffraction/scattering method ranging from 0.1 to 50 μm, and in which the primary particle is in a spherical form, can be preferably blended. In addition, the silicone elastomer constituting the silicone elastomer powders may have a hardness preferably not exceeding 80, and more preferably not exceeding 65, when measured by means of a type A durometer according to JIS K 6253 “Method for determining hardness of vulcanized rubber or thermoplastic rubber”.

The silicone elastomer powders may be subjected to a surface treatment with a the organosilane (I), silicone resin, silica or the like. As examples of the aforementioned surface treatments, mention may be made of, for example, those described in Japanese Unexamined Patent Application, First Publication No. H02-243612; Japanese Unexamined Patent Application, First Publication No. H08-12545; Japanese Unexamined Patent Application, First Publication No. H08-12546; Japanese Unexamined Patent Application, First Publication No. H08-12524; Japanese Unexamined Patent Application, First Publication No. H09-241511; Japanese Unexamined Patent Application, First Publication No. H10-36219; Japanese Unexamined Patent Application, First Publication No. H11-193331; Japanese Unexamined Patent Application, First Publication No. 2000-281523 and the like. As the silicone elastomer powders, crosslinking silicone powders listed in “Japanese Cosmetic Ingredients Codex (JCIC)” correspond thereto. As commercially available products of the silicone elastomer powders, there are Trefil E-506S, Trefil E-508, 9701 Cosmetic Powder, and 9702 Powder, manufactured by Dow Corning Toray Co., Ltd., and the like. As examples of the surface treatment agents, include, but are not limited to, the organosilane (I), methylhydrogenpolysiloxane, silicone resins, metallic soap, silane coupling agents, inorganic oxides such as silica, titanium oxide and the like and fluorine compounds such as perfluoroalkylsilane, perfluoroalkyl phosphoric ester salts and the like.

Pulverulent inorganic or organic fillers can also be added, generally in an amount by weight from 0 to 40% with respect to the weight of the personal care composition. These pulverulent fillers can be chosen from talc, micas, kaolin, zinc or titanium oxides, calcium or magnesium carbonates, silica, spherical titanium dioxide, glass or ceramic beads, metal soaps derived from carboxylic acids having 8-22 carbon atoms, non-expanded synthetic polymer powders, expanded powders and powders from natural organic compounds, such as cereal starches, which may or may not be crosslinked. The fillers may typically be present in a proportion of from 0 to 35% of the total weight of the composition, more typically 5 to 15%. Mention may be made in particular of talc, mica, silica, kaolin, nylon powders (in particular ORGASOL), polyethylene powders, Teflon, starch, boron nitride, copolymer microspheres such as EXPANCEL (Nobel Industrie), polytrap and silicone resin microbeads (TOSPEARL from Toshiba, for example). In one embodiment, the pulverulent filler is an inorganic filler, alternatively zinc or titanium oxides.

The personal care compositions may include a sunscreen. Sunscreens typically absorb ultraviolet light between 290-320 nanometers (the UV-B region) such as, but not exclusively, para-aminobenzoic acid derivatives and cinnamates such as octyl methoxycinnamate and those which absorb ultraviolet light in the range of 320-400 nanometers (the UV-A region) such as benzophenones and butyl methoxy dibenzoylmethane. Some additional examples of sunscreens are 2-ethoxyethyl p-methoxycinnamate; menthyl anthranilate; homomethyl salicylate; glyceryl p-aminobenzoate; isobutyl p-aminobenzoate; isoamyl p-dimethylaminobenzoate; 2-hydroxy-4-methoxybenzophenones sulfonic acid; 2,2′-dihydroxy-4-methoxybenzophenone; 2-hydroxy-4-methoxybenzophenone; 4-mono and 4-bis(3-hydroxy-propyl)amino isomers of ethyl benzoate; and 2-ethylhexyl p-dimethylaminobenzoate. In various embodiments, the sunscreen is as described in EP-A-678,292, which is expressly incorporated herein by reference in one or more non-limiting embodiments. In various embodiments, sunscreens include at least one carboxylic or better still sulphonic acid radical. This acid radical can be in free form or in partially or totally neutralized form. It is possible to use one or more hydrophilic screening agents containing acid functionality. As examples of acidic screening agents containing at least one SO₃H group, mention may be made more particularly of 3-benzylidine-2-camphorsulphonic derivatives. A particularly typical compound is benzene-1,4-[di(3-methylidenecamphor-10-sulphonic acid)]. This screening agent is a broad-band screening agent capable of absorbing ultraviolet rays with wavelengths of between 280 nm and 400 nm, with absorption maxima of between 320 nm and 400 nm, in particular at 345 nm. It is used in acid form or salified with a base chosen from triethanolamine, sodium hydroxide and potassium hydroxide. In addition, it can be in cis or trans form. This screening agent is known under the trade name Mexoryl SX. Other specific examples are 4-(3-methylidenecamphor)benzenesulphonic acid, 3-benzylidenecamphor-10-sulphonic acid, 2-methyl-5-(3-methylidenecamphor)benzenesulphonic acid, 2-chloro-5-(3-methylidenecamphor)benzenesulphonic acid, 3-(4-methyl)benzylidenecamphor-10-sulphonic acid, (3-t-butyl-2-hydroxy-5-methyl)benzylidenecamphor-10-sulphonic acid, (3-t-butyl-2-hydroxy-5-methoxy)benzylidenecamphor-10-sulphonic acid, (3,5-di-tert-butyl-4-hydroxy)benzylidenecamphor-10-sulphonic acid, 2-methoxy-5-(3-methylidenecamphor)benzenesulphonic acid, 3-(4,5-methylenedioxy)benzylidenecamphor-10-sulphonic acid, 3-(4-methoxy)benzylidenecamphor-10-sulphonic acid, 3-(4,5-dimethoxy)benzylidenecamphor-10-sulphonic acid, 3-(4-n-butoxy)benzylidenecamphor-10-sulphonic acid, 3-(4-n-butoxy-5-methoxy)benzylidenecamphor-10-sulphonic acid, 2-[4-(camphormethylidene)phenyl]benzimidazole-5-sulphonic acid. Suitable compounds are described in U.S. Pat. No. 4,585,597, and FR 2,236,515, 2,282,426, 2,645,148, 2,430,938 and 2,592,380, each of which is expressly incorporated herein by reference in one or more non-limiting embodiments. The screening agent containing a sulphonic group can also be a sulphonic derivative of benzophenone or 2-phenylbenzimidazole-5-sulphonic acid, having excellent photoprotective power in the UV-B radiation range and is sold under the trade name “Eusolex 232” by Merck, benzene-1,4-di(benzimidazol-2-yl-5-sulphonic acid), benzene-1,4-di(benzoxazol-2-yl-5-sulphonic acid). The hydrophilic screening agent(s) can be present in the final composition according to the disclosure in a content which can be from 0.1 to 20%, typically from 0.2 to 10%, by weight relative to the total weight of the personal care composition.

Additional lipophilic screening agents can be utilized such as those derived from dibenzoylmethane and more especially 4-tert-butyl-4′-methoxydibenzoylmethane, which effectively have a high intrinsic power of absorption. These dibenzoylmethane derivatives, which are products that are well known per se as UV-A active screening agents, are described in particular in French patent applications FR-A-2,326,405 and FR-A-2,440,933, as well as in European patent application EP-A-0,114,607, each of which is expressly incorporated herein by reference in one or more non-limiting embodiments. 4-(tert-butyl)-4′-methoxydibenzoylmethane is currently sold under the trade name “Parsol 1789” by Givaudan. Another dibenzoylmethane derivative which is typical according to the present disclosure is 4-isopropyldibenzoylmethane, this screening agent being sold under the name “Eusolex 8020” by Merck. Similarly octocrylene, a liquid lipophilic screening agent that is already known for its activity in the UV-B range is commercially available, and is sold in particular under the name “Uvinul N 539” by BASF. As another lipophilic (or liposoluble) screening agent which can be used in the disclosure, mention may also be made of p-methylbenzylidenecamphor, which is also known as a UV-B absorber and is sold in particular under the trade name “Eusolex 6300” by Merck. The lipophilic screening agent(s) can be present in the composition according to the disclosure in a content which can be from 0.5 to 30%, typically from 0.5 to 20%, of the total weight of the composition. Other examples of lipophilic or hydrophilic organic screening agents are described in patent application EP-A-0,487,404, which is expressly incorporated herein by reference in one or more non-limiting embodiments. The cosmetic and/or dermatological compositions according to the disclosure can also include pigments or alternatively nanopigments (average primary particle size: generally between 5 nm and 100 nm, typically between 10 and 50 nm) of coated or uncoated metal oxides, such as, for example, nanopigments of titanium oxide (amorphous or crystallized in rutile and/or anatase form), of iron oxide, of zinc oxide, of zirconium oxide or of cerium oxide, which are all photoprotective agents that are well known per se and which act by physically blocking (reflection and/or scattering) UV radiation. Standard coating agents are, moreover, alumina and/or aluminium stearate, and silicones. Such coated or uncoated metal oxide nanopigments are described in particular in patent applications EP-A-0,518,772 and EP-A-0,518,773, each of which is expressly incorporated herein by reference in one or more non-limiting embodiments.

A thickening agent may be utilized in the personal care composition to provide a convenient viscosity. For example, viscosities of from 500 to 25,000 mm²/s at 25° C. or more alternatively of from 3,000 to 7,000 mm²/s at 25° C. may be obtained. Suitable thickening agents are exemplified by sodium alginate, gum arabic, polyoxyethylene, guar gum, hydroxypropyl guar gum, ethoxylated alcohols, such as laureth-4 or polyethylene glycol 400, cellulose derivatives exemplified by methylcellulose, methylhydroxypropylcellulose, hydroxypropylcellulose, polypropylhydroxyethylcellulose, starch, and starch derivatives exemplified by hydroxyethylamylose and starch amylose, locust bean gum, electrolytes exemplified by sodium chloride and ammonium chloride, and saccharides such as fructose and glucose, and derivatives of saccharides such as PEG-120 methyl glucose diolate or combinations of 2 or more of these. Alternatively the thickening agent is selected from cellulose derivatives, saccharide derivatives, and electrolytes, or from a combination of two or more of the above thickening agents exemplified by a combination of a cellulose derivative and any electrolyte, and a starch derivative and any electrolyte. The thickening agent, where used is present in a shampoo composition, may provide a viscosity of from 500 to 25,000 mm²/s at 25° C. Alternatively the thickening agent may be present in an amount from 0.05 to 10 wt % and alternatively 0.05 to 5 wt % based on the total weight of the personal care composition.

Stabilizing agents can also be used, e.g. in a water phase of an emulsion. Suitable water phase stabilizing agents can include alone or in combination one or more electrolytes, polyols, alcohols such as ethyl alcohol, and hydrocolloids. Typical electrolytes are alkali metal salts and alkaline earth salts, especially the chloride, borate, citrate, and sulfate salts of sodium, potassium, calcium and magnesium, as well as aluminum chlorohydrate, and polyelectrolytes, especially hyaluronic acid and sodium hyaluronate. When the stabilizing agent is, or includes, an electrolyte, it amounts to 0.1 to 5 wt % and more alternatively 0.5 to 3 wt % of the personal care composition. The hydrocolloids include gums, such as Xantham gum or Veegum and thickening agents, such as carboxymethyl cellulose. Polyols, such as glycerine, glycols, and sorbitols can also be used. Alternative polyols are glycerine, propylene glycol, sorbitol, and butylene glycol. If a large amount of a polyol is used, one need not add the electrolyte. However, it is typical to use a combination of an electrolyte, a polyol and a hydrocolloid to stabilize the water phase, e.g. magnesium sulfate, butylene glycol and Xantham gum.

Referring back, the composition may be an anti-perspirant and deodorant compositions under but not limited to the form of sticks, soft solid, roll on, aerosol, and pumpsprays. Some examples of antiperspirant agents and deodorant agents are Aluminum Chloride, Aluminum Zirconium Tetrachlorohydrex GLY, Aluminum Zirconium Tetrachlorohydrex PEG, Aluminum Chlorohydrex, Aluminum Zirconium Tetrachlorohydrex PG, Aluminum Chlorohydrex PEG, Aluminum Zirconium Trichlorohydrate, Aluminum Chlorohydrex PG, Aluminum Zirconium Trichlorohydrex GLY, Hexachlorophene, Benzalkonium Chloride, Aluminum Sesquichlorohydrate, Sodium Bicarbonate, Aluminum Sesquichlorohydrex PEG, Chlorophyllin-Copper Complex, Triclosan, Aluminum Zirconium Octachlorohydrate, and Zinc Ricinoleate.

The personal care compositions can be an aerosol in combination with propellant gases, such as carbon dioxide, nitrogen, nitrous oxide, volatile hydrocarbons such as butane, isobutane, or propane and chlorinated or fluorinated hydrocarbons such as dichlorodifluoromethane and dichlorotetrafluoroethane or dimethylether.

Silicone compositions may also be included in the personal care compositions. For example, such silicones include silicone fluids, gums, resins, elastomers; silicone surfactants and emulsifiers such as silicone polyethers, organofunctional silicones such as amino functional silicones and alkylmethylsiloxanes. Alkylmethylsiloxanes may be included in the present compositions. These siloxane polymers generally typically have the formula Me₃SiO[Me₂SiO]_{y}[MeRSiO]_{z}SiMe₃, in which R is a hydrocarbon group containing 6-30 carbon atoms, Me represents methyl, and the degree of polymerization (DP), i.e., the sum of y and z is 3-50. Both the volatile and liquid species of alkylmethysiloxanes can be used in the composition.

Silicone gums other than those described above may also be included in the personal care compositions. Suitable non-limiting gums include insoluble polydiorganosiloxanes having a viscosity in excess of 1,000,000 centistoke (mm²/s) at 25° C., alternatively greater than 5,000,000 centistoke (mm²/s) at 25° C. These silicone gums are typically sold as compositions already dispersed in a suitable solvent to facilitate their handling. Ultra-high viscosity silicones can also be included as optional ingredients. These ultra-high viscosity silicones typically have a kinematic viscosity greater than 5 million centistoke (mm²/s) at 25° C. up to 20 million centistoke (mm²/s) at 25° C. Compositions of this type in are described for example in U.S. Pat. No. 6,013,682, which is expressly incorporated herein by reference in one or more non-limiting embodiments.

Silicone resins may also be included in the personal care composition. These resins are generally highly crosslinked polymeric siloxanes. Crosslinking is typically obtained by incorporating trifunctional and/or tetrafunctional silanes with the monofunctional silane and/or difunctional silane monomers used during manufacture. The degree of crosslinking required to obtain a suitable silicone resin will vary according to the specifics of silane monomer units incorporated during manufacture of the silicone resin. In general, any silicone having a sufficient level of trifunctional and tetrafunctional siloxane monomer units, and hence possessing sufficient levels of crosslinking to dry down to a rigid or a hard film can be used. Commercially available silicone resins suitable for applications herein are generally supplied in an unhardened form in low viscosity, volatile or nonvolatile silicone fluids. The silicone resins may be incorporated into compositions of the disclosure in their non-hardened forms rather than as hardened resinous structures.

Silicone carbinol fluids may be included in the personal care composition. These materials can be commonly described as substituted hydrocarbyl functional siloxane fluids or resins and some are described in WO 03/101412 A2, which is expressly incorporated herein by reference in one or more non-limiting embodiments.

Water soluble or water dispersible silicone surfactants may also be included in the personal care compositions that are water-based or oil-in-water emulsions. In addition, water insoluble silicon surfactants may also be included in oil-based or water-in-oil type dispersion formulations These are also known as polyalkylene oxide silicone copolymers, silicone poly(oxyalkylene) copolymers, silicone glycol copolymers, monoglycerol functional silicones, diglycerol functional silicones, triglycerol silicones, polyglycerol silicons, or silicone surfactants. These can be linear rake or graft type materials, or ABA type where the B is the siloxane polymer block, and the A is the poly(oxyalkylene) group. The poly(oxyalkylene) group can consist of polyethylene oxide, polypropylene oxide, or mixed polyethylene oxide/polypropylene oxide groups. Other oxides, such as butylene oxide or phenylene oxide are also possible.

The personal care composition may also include a solvent such as (i) organic compounds, (ii) compounds containing a silicon atom, (iii) mixtures of organic compounds, (iv) mixtures of compounds containing a silicon atom, or (v) mixtures of organic compounds and compounds containing a silicon atom; used on an industrial scale to dissolve, suspend, or change the physical properties of other materials.

In general, the organic compounds are aromatic hydrocarbons, aliphatic hydrocarbons, alcohols, aldehydes, ketones, amines, esters, ethers, glycols, glycol ethers, alkyl halides, or aromatic halides. Representative of some common organic solvents are alcohols such as methanol, ethanol, 1-propanol, cyclohexanol, benzyl alcohol, 2-octanol, ethylene glycol, propylene glycol, and glycerol; aliphatic hydrocarbons such as pentane, cyclohexane, heptane, VM&P solvent, and mineral spirits; alkyl halides such as chloroform, carbon tetrachloride, perchloroethylene, ethyl chloride, and chlorobenzene; amines such as isopropylamine, cyclohexylamine, ethanolamine, and diethanolamine; aromatic hydrocarbons such as benzene, toluene, ethylbenzene, and xylene; esters such as ethyl acetate, isopropyl acetate, ethyl acetoacetate, amyl acetate, isobutyl isobutyrate, and benzyl acetate; ethers such as ethyl ether, n-butyl ether, tetrahydrofuran, and 1,4-dioxane; glycol ethers such as ethylene glycol monomethyl ether, ethylene glycol monomethyl ether acetate, diethylene glycol monobutyl ether, and propylene glycol monophenyl ether; ketones such as acetone, methyl ethyl ketone, cyclohexanone, diacetone alcohol, methyl amyl ketone, and diisobutyl ketone; petroleum hydrocarbons such as mineral oil, gasoline, naphtha, kerosene, gas oil, heavy oil, and crude oil; lubricating oils such as spindle oil and turbine oil; and fatty oils such as corn oil, soybean oil, olive oil, rape seed oil, cotton seed oil, sardine oil, herring oil, and whale oil.

Other miscellaneous organic solvents can also be used, such as acetonitrile, nitromethane, dimethylformamide, propylene oxide, trioctyl phosphate, butyrolactone, furfural, pine oil, turpentine, and m-creosol.

Solvents may also include volatile flavoring agents such as oil of wintergreen; peppermint oil; spearmint oil; menthol; vanilla; cinnamon oil; clove oil; bay oil; anise oil; eucalyptus oil; thyme oil; cedar leaf oil; oil of nutmeg; oil of sage; cassia oil; cocoa; licorice; high fructose corn syrup; citrus oils such as lemon, orange, lime, and grapefruit; fruit essences such as apple, pear, peach, grape, strawberry, raspberry, cherry, plum, pineapple, and apricot; and other useful flavoring agents including aldehydes and esters such as cinnamyl acetate, cinnamaldehyde, eugenyl formate, p-methylanisole, acetaldehyde, benzaldehyde, anisic aldehyde, citral, neral, decanal, vanillin, tolyl aldehyde, 2,6-dimethyloctanal, and 2-ethyl butyraldehyde.

Moreover, solvents may include volatile fragrances such as natural products and perfume oils. Some representative natural products and perfume oils are ambergris, benzoin, civet, clove, leaf oil, jasmine, mate, mimosa, musk, myrrh, orris, sandalwood oil, and vetivert oil; aroma chemicals such as amyl salicylate, amyl cinnamic aldehyde, benzyl acetate, citronellol, coumarin, geraniol, isobornyl acetate, ambrette, and terpinyl acetate; and the various classic family perfume oils such as the floral bouquet family, the oriental family, the chypre family, the woody family, the citrus family, the canoe family, the leather family, the spice family, and the herbal family.

This disclosure also provides methods for forming personal care compositions. The methods include combining the organosilane in a personal care composition. In one embodiment, the organosilane is prepared individually and then combined later with the personal care composition ingredients. Techniques known in the art for formation of personal care formulations, including but not limited to, mixing techniques, cold blends or application of heat to facilitate forming the composition, can be used. The order of addition used herein can be any known in the art.

The paint composition, antifog composition, an ink composition may comprise ingredients common to such compostions including organic and/or inorganic colorants, pigments, polymers, solvents and diluents, and dyes including, but not limited to those described above for the personal care composition. These compostions can be made by methods known in the art using standard equipment and conditions known in the are such as the equipment and methods described for the personal care formulations above.

### Coating and Surface Treating Compositions

The coating composition and surface treating composition may comprise ingredients, in addition to the organosilane, that are common to such compostions including, but not limited to, solvents, diluents, dispersants, and polymers. Examples of such ingredients include, but are not limited to, those described for the personal care composition above. In one embodiment, the coating composition or surface treating composition comprises, a surface treated powder.

The composition my comprise and antifouling agent. Antifouling agents are materials that have a killing or repelling effect against aquatic fouling organisms. Examples include inorganic and organic antifoulants.

Examples of inorganic antifouling agents include, but are not limited to, cuprous oxide, copper thocyanate (general name: copper rhodanide), cupronickel, and copper powder. Among these, cuprous oxide and copper rhodanide are particularly preferred.

Examples of the organic antifoulants include: organic copper compounds such as 2-mercaptopyridine-N-oxide copper (general name: copper pyrithione) and the like; organic zinc compounds such as 2-mercaptopyridine-N-oxide zinc (general name: zinc pyrithione), zinc ethylene bis(dithio carbamate) (general name: zineb), zinc bis(dimethyldithiocarbamate)(general name: ziram), dizinc bis(dimethyldithiocarbamate)ethylenebis(dithiocarbamate) (general name: polycarbamate) and the like; organic boron compounds such as pyridine-triphenylborane, 4-isopropyl pyridyl-diphenylmethyl borane, 4-phenyl pyridiyl-diphenyl borane, triphenylboron-n-octadecyl amine, triphenyl[3-(2-ethylhexyloxy) propyl amine]boron and the like; maleimide compounds such as 2,4,6-trichloromaleimide, N-(2,6-diethylphenyl)-2,3-dichloromaleimide and the like; and 4,5-dichloro-2-n-octyl-3-isothiazolone (general name: Sea-Nine 211), 3,4-dichlorophenyl-N—N-dimethylurea (general name: diuron), 2-methylthio-4-t-butylamino-6-cyclopropylamino-s-triazine (general name: Irgarol 1051), 2,4,5,6-tetrachloroisophthalonitrile (general name: chlorothalonil), N-dichlorofluoromethylthio-N′,N′-dimethyl-N-p-tolylsulfamide (general name: tolylfluanid), N-dichloromethylthio-N′,N′-dimethyl-N-phenylsulfamide (general name: dichlofluanid), 2-(4-thiazolyl)benzimidazole (general name: thiabendazole), 3-(benzo [b]thien-2-yl)-5,6-dihydro-1,4,2-oxathiazine-4-oxide (general name: bethoxazine), 2-(p-chlorophenyl)-3-cyano-4-bromo-5-trifluoromethyl pyrrole (general name: ECONEA 028), etc. Among them, particularly preferred are zinc pyrithione, copper pyrithione, pyridine-triphenylborane, 4-isopropyl pyridyl-diphenylmethyl borane, bethoxazine, zineb, Sea-Nine 211, and Irgarol 1051. More preferred are copper pyrithione, zinc pyrithione, pyridine-triphenylborane, and bethoxazine.

As the antifoulant, preferred are cuprous oxide, copper rhodanide, zinc pyrithione, copper pyrithione, pyridine-triphenylborane, 4-isopropyl pyridyl-diphenylmethyl borane, bethoxazine, zineb, Sea-Nine 211, Irgarol 1051, tolylfluanid, and dichlofluanid. More preferred are cuprous oxide, copper pyrithione, zinc pyrithione, pyridine-triphenylborane, and Sea-Nine 211. These antifoulants may be used singly or in combination.

The amount of the antifoulant in the composition of the present invention is not particularly limited, and is usually from 0.1 to 75% by mass, and preferably from 1 to 60% by mass with respect to the solid content of the composition of the present invention. When the amount of the antifoulant is less than 0.1% by mass, a sufficient antifouling effect might not be obtained. When the amount of the antifoulant is over 75% by mass, the obtained coating film is fragile, and adherence of the coating film to the coated object is weak, and thus the coating film does not sufficiently exhibit the function as an antifouling coating film.

A typical antifouling coating may comprise:

(A) One or more substrates or surfaces described below, (B) (optionally) an organopolysiloxane, silsequioxane, or silicone resin, where the organopolysiloxane, silsequioxane, or silicone resin has two or more silanol or alkoxy groups per 1 molecule, (C) the organosilane (I) described above, preferably where the organosilane has two or three hydrolysable groups in 1 molecule, and/or the partial hydrolysis-condensation product or the organosilane (I) described above, (D) a metal curing catalyst, (E) an antifouling agent described above, and (F) a volatile solvent selected from alcohol or hydrocarbon or ester or ether.

In the present invention, a different curing catalyst (D) may be added to the antifouling coating composition, if necessary, when the advantageous effects of the present invention are not lowered. Specific examples include carboxylic acids such as acetic acid, propionic acid, butyric acid, 2-ethylhexanoic acid, lauric acid, stearic acid, oleic acid, linoleic acid, pivalic acid, 2,2-dimethylbutyric acid, 2,2-diethylbutyric acid, 2,2-dimethylhexanoic acid, 2,2-diethylhexanoic acid, 2,2-dimethyloctanoic acid, 2-ethyl-2,5-dimethylhexanoic acid, neodecanoic acid, versatic acid and other acids; derivatives of the carboxylic acids (carboxylic anhydrides, esters, amides, nitriles and acyl chlorides); metal carboxylates such as tin carboxylate, lead carboxylate, bismuth carboxylate, potassium carboxylate, calcium carboxylate, barium carboxylate, titanium carboxylate, zirconium carboxylate, hafnium carboxylate, vanadium carboxylate, manganese carboxylate, iron carboxylate, cobalt carboxylate, nickel carboxylate, cerium carboxylate and other carboxylates; titanium compounds such as tetrabutyl titanate, tetrapropyl titanate, titanium tetrakis(acetylacetonate), bis(acetylacetonate)diisopropoxytitanium, diisopropoxytitanium bis(ethylacetonate) and other titanates; organictin compounds such as dibutyltin dilaurate, dibutyltin maleate, dibutyltin phthalate, dibutyltin dioctanoate, dibutyltin bis(2-ethylhexanoate), dibutyltin bis(methylmaleate), dibutyltin bis(ethylmaleate), dibutyltin bis(butylmaleate), dibutyltin bis(octylmaleate), dibutyltin bis(tridecylmaleate), dibutyltin bis(benzylmaleate), dibutyltin diacetate, dioctyltin bis(ethylmaleate), dioctyltin bis(octylmaleate), dibutyltin dimethoxide, dibutyltin bis(nonylphenoxide), dibutenyltin oxide, dibutyltin oxide, dibutyltin bis (acetylacetonate), dibutyltin bis(ethylacetoacetonate), a reactant of dibutyltin oxide and a silicate compound, and a reactant of dibutyltin oxide and a phthalic acid ester and other tin compounds; aluminum compounds such as aluminum tris(acetylacetonate), aluminum tris(ethylacetoacetate), and diisopropoxyaluminum methylacetoacetate and other aluminum compounds; zirconium compounds such as zirconium tetrakis(acetylacetonate) and the like; various metal alkoxides such as tetrabutoxyhafnium and the like; organic acidic phosphates; organic sulfonic acids such as trifluoromethanesulfonic acid and the like; and inorganic acids such as hydrochloric acid, phosphoric acid, boronic acid and other acids; Lewis acids such as halogenated metal compounds including aluminum chloride, titanium chloride, zirconium chloride, zinc chloride, zinc bromide, iron chloride, copper chloride, antimony chloride, tin chloride and the like; triflates including metal triflates such as indium triflate, tin triflate, trialkylsilyltriflate and the like; and the derivatives thereof.

When any one of these curing catalysts is used together, the catalyst activity becomes high and an improvement of resultant cured products is expected in depth curability, thin-layer curability, adhesiveness, and others. However, if the amount of the added carboxylic acid is large, a sufficient adhesiveness of resultant cured products may not be obtained.

An anti-fog agent may be the oganosilane (1) in combination with any type of silica. For example, the composition may comprise the organosilane (1) and a colloidal silica-sol.

The coating or treating composition may be used to coat surfaces including, but not limited to, is skin, hair, textile, fiber, inorganic powder, organic powders, wall, floor, glass, mirror, or metal. The treating composition may be used to treat inorganic powders. Methods of treating powders are generally known in the art. For example, methods of treating powders are disclosed in US20140323590 A1, which is hereby incorporated by reference for its disclosure related to surface treating methods. US20-140323590 A1 discloses methods such as mixing a excess amount of a powder surface treatment agent comprising a modified organopolysiloxane with a powder, and mixing the surface treating agent and powder with a siloxane and zirconia beads in a paint shaker for an hour to create a dispersion of the surface treated powder in siloxane.

A method of coating or treating a surface comprising applying to the surface the treating or coating composition. A method of coating a surface comprising applying the coating composition to a surface.

Another embodiment of the invention is a treatment composition comprising the product of the hydrolysis and/or condensation of the organosilane of formula (I) or of the organosilane produced by method A of preparing an organosilane or Method B for preparing the organosilane. The treatment composition further comprises at least one additional ingredient. Examples of the at least one additional ingredient include, but are not limited to, those described above for the personal care composition.

On embodiment of the invention is a hydrophilized substrate wherein the substrate has been treated or coated with the treating or coating composition.

Examples of the hydrophilized substrate include, but are not limited to, a powder, alternatively a metal oxide; glass; pigment; ketatinous materials, alternatively skin, alternatively hair; fabrics, alternatively wool, nylon, or rayon, alternatively wool treated with the treating or coating composition. Examples of the metal oxide include, but are not limited to, zinc oxide or titanium dioxide. Zinc oxide and titanium dioxide are available commercially.

### EXAMPLES

The invention is further illustrated by, and an invention embodiment may include any combinations of features and limitations of, the non-limiting examples thereof that follow.

The following examples are included to demonstrate particular embodiments of the invention. It should be appreciated by those of skill in the art that the techniques disclosed in the examples which follow represent techniques discovered by the inventor to function well in the practice of the invention, and thus can be considered to constitute modes for its practice.

However, those of skill in the art should, in light of the present disclosure, appreciate that many changes can be made in the specific embodiments which are disclosed and still obtain a like or similar result without departing from the spirit and scope of the invention. Unless otherwise indicated, all percentages are in weight % (wt. %). Ambient temperature is about 23° C. unless indicated otherwise.

### Example 1

Process for Treatment of Zinc Oxide with Silane: 10 grams of a 20% (w/w) mixture of zinc oxide (MZ-500 from Tayca Corporation having an average particle size of 25 nanometers) in isopropyl alcohol (IPA) was slowly added to a mixture of 40 g of zinc oxide, 75 g of IPA, and 0.1 g of water in a glass bottle with mixing by a dispersing blade rotating at 1100-1200 rpm and mixed well until a rough dispersion was formed. The glass bottle was then sealed and subjected to ultrasonic for 30 min. IPA was then removed from the sample to produce a dried aggregate. The aggregate was then milled to obtain a silane-treated zinc oxide fine powder. This silane-treated zinc oxide fine powder was then formed into a 35% aqueous dispersion.

Preparation of 35% Aqueous Dispersion of Treated Zinc Oxide: 65 g of water and 35 g of the fine powder of silane-treated zinc oxide were added to a 225 milliliter glass bottle then mixed by hand to form a rough slurry. Zirconia beads (0.8 mm diameter) were then added to the bottle. The bottle was then sealed and shaken on a paint shaker for 15 hours to form a dispersion. The zirconia beads where then removed by filtration through a suitable mesh screen.

Testing of Silane-Treated Zinc Oxide Dispersions for Transmittance and Dispersibility:

| | Description (35% dispersions prepared as described |
|---|---|
| Sample No. | above) |
| Control | Control. Dispersion of zinc oxide with no silane treatment. |
| B-1 | Diglycerolpropyltriethoxysilane treated zinc oxide |
| | dispersion. |
| B-2 | Diglycerolpropylmethyldimethoxysilane treated zinc |
| | oxide dispersion |
| B-3 | Methyl -PEG-7-propylmethyldimethoxysilane treated zinc |
| | oxide dispersion |
| B-4 | Monoglycerolpropylmethyldimethoxysilane treated zinc |
| | oxide |
| B-5 | Methyltrimethoxysilane treated zinc oxide |
| Control | No treatment |

| | Control | B-1 | B-2 | B-4 | B-3 | B-5 |
|---|---|---|---|---|---|---|
| Vis*¹ | 2440 | 568 | 166 | 1210 | 2340 | Gel/ |
| | | | | | | Separation |
| D(v, 0.5), nm*² | 102 | 68 | 53 | 74 | 98 | 6413 |
| D(v, 0.9), nm*² | 182 | 117 | 89 | 123 | 165 | 8649 |
| T %@550 nm*³ | 6.3 | 71.8 | 92.5 | 64.0 | 43.6 | 58.4 |
| T %@350 nm | 0.4 | 0.1 | 0.5 | 0.1 | 0.0 | 0.0 |
| (UV-A)*³ | | | | | | |
| T %@300 nm | 0.6 | 0.0 | 0.2 | 0.0 | 0.0 | 0.0 |
| (UV-B)*³ | | | | | | |

*¹35% dispersion;
*²Dilute aqueous solution of 35% dispersion with ultrasonic;
*³1.063 mm thin liquid film of 0.25% dispersion of 1,3-BG (equals to 2.7 micrometers as solid)

### Example 2

Glass Coating Properties: Glass substrates were prepared by cleaning the glass by dipping ain an alkaline medium followed by rinsing with deionized water and drying at 23° C. The glass was then dipped in an acid followed by drying at 23° C. Next the glass was dip coated with a 0.6% (w/w) solution of silane in ethanol. The silane-treated glass was then cured at 130° C. for 2 hours, cleaned with deionized water and gently wiped dry. The surfaces were then tested for contact angle with a 10 microliter drop of water. The silanes tested and the results are in the following tables.

| Coating No. | Silane in Film |
|---|---|
| 1a | Diglycerolpropylmethyldimethoxysilane |
| 1b | Diglycerolpropyltriethoxysilane |
| 2 | Monoglycerolpropylmethyldimethoxysilane |
| 3 | Methyl-PEG-7-propylmethyldimethoxysilane |
| | ((CH₃O)₃(CH₃)Si(CH₂)₃O(CH₂CH₂O)₇CH₃) |
| 4 | Methyltrimethoxysilane |

### Contact Angle Test Results:

| Coating | 1a | 1b | 2 | 3 | 4 |
|---|---|---|---|---|---|
| Contact | 48° | 41° | 53° | 49° | 84° |
| Angle | (s = 3.3°) | (s = 3.1°) | (s = 4.6°) | (s = 2.2°) | (s = 3.3°) |

1. A composition, the composition comprising:
an organosilane having formula (I)
X-A-Z, (I)
wherein X is —SiR⁴ₙR²₍₃₋ₙ₎, wherein each R⁴ is independently OR¹ or halogen, wherein each R¹ in independently hydrogen or C₁₋₄ hydrocarbyl and each R² is independently C₁₋₄ hydrocarbyl, and n is from 1 to 3, A is C₁₋₁₀ alkylene, wherein the alkylene backbone of A is substituted with one oxygen atom, one nitrogen atom, —NC(O)O—, or —NC(O)N— or wherein A is a substituted C₁₋₁₀ hydrocarbylene represented by the following structure 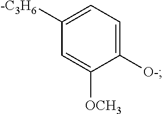
and Z is a diglycerol group or a polyglycerol group, and
wherein the composition is a personal care composition, surface treating composition, an antifog composition, a coating composition, a surface treated powder, a paint composition, or an ink composition.

2. The composition of claim 1, wherein Z is represented by Glyₐ, wherein Gly is R³CH₂CH(R³)CH₂R³, where each R³ independently represents hydroxyl, an oxygen atom linking to A, or an oxygen atom linking to another Gly unit, and a is an integer from 2 to 6.

3. (canceled)

4. The composition of claim 1, wherein A is substituted with —OH or —CH₂OH and Z is N(R⁷)(CH3)CH2[C(H)(R⁶)]₄CH₂(R⁶), wherein each R⁶ independently represents hydroxyl or an oxygen atom linking to A, and R⁷ represents a hydrogen atom, hydrocarbyl, or a bond to A

5. (canceled)

6. A method of treating a surface comprising: applying a composition to a surface, where the composition comprises:
an organosilane having formula (I)
X-A-Z, (I)
wherein X is —SiR⁴ₙR²₍₃₋ₙ₎, wherein each R⁴ is independently OR¹ or halogen, wherein each R¹ in independently hydrogen or C₁₋₄ hydrocarbyl and each R² is independently C₁₋₄ hydrocarbyl, and n is from 1 to 3, A is C₁₋₁₀ alkylene, wherein the alkylene backbone of A is substituted with one oxygen atom, one nitrogen atom, —NC(O)O—, or —NC(O)N— or wherein A is a substituted C₁₋₁₀ hydrocarbylene represented by the following structure 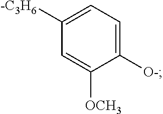
and Z is a diglycerol group or a polyglycerol group, and wherein the composition is a personal care composition, surface treating composition, an antifog composition, a coating composition, a surface treated powder, a paint composition, or an ink composition.

7. The method of claim 6, wherein the surface is skin, hair, textile, fiber, inorganic powder, wall, floor, glass, mirror, or metal.

8. The method of claim 7, wherein the surface is an inorganic powder.

9. The method of claim 8, wherein the powder a ZnO or TiO₂ powder.

10. The composition of claim 1, further comprising at least one of the following materials a) through n): a) a sunscreen, b) a colorant, c) an anti-fog additive, d) a polymer other than organosilane, e) a surfactant, f) an antifouling agent, g) a filler h) a powder, i) a cosmetic ingredient, j) an oil, k) a wax, l) water, m) another organosilane, n) a water-soluble material, and o) an elastomer.

11. The composition according to claim 10, wherein the composition is a dispersion.

12. The composition according to claim 10, wherein the composition comprises d), and d) is a polysiloxane.

13. The composition of claim 10, comprising g), h), or g) and h) wherein g), h), or g) and h) are surface treated with the organosilane.

14. The composition according to claim 13, wherein g), h), or g) and h) are dispersed in the composition.

15. The composition of claim 10, wherein the composition is a sunscreen composition.
